(19) United States
(12) Patent Application Publication
(10) Pub. No.: US 2016/0015814 A1
(43) Pub. Date: Jan. 21, 2016

(54) Fast-acting insulin formulation comprising a substituted anionic compound and a polyanionic compound

(71) Applicant: ADOCIA, Lyon (FR)

(72) Inventors: Olivier SOULA, Meyzieu (FR); Richard CHARVET, Rillieux la Pape (FR); Guilhem MORA, Lyon (FR)

(21) Appl. No.: 14/711,378

(22) Filed: May 13, 2015

(30) Foreign Application Priority Data
May 14, 2014 (FR) 14/54315

(51) Int. Cl. A61K 47/26; A61K 47/12; A61K 38/28

(52) U.S. Cl. CPC A61K 47/26; A61K 38/28; A61K 47/12

(57) ABSTRACT

A composition, in aqueous solution, comprising insulin in hexameric form, at least one substituted anionic compound and at least one polyanionic compound, said substituted anionic compound consisting of a discrete number n of between 1 and 8 of identical or different saccharide units, linked via identical or different glycoside bonds, said saccharide unit or one of said saccharide units being in open, oxidized or reduced form, said compound comprising salifiable carboxyl groups and said substituted anionic compound bearing on its reductive chain end at least one radical AA resulting from an aromatic amino acid comprising a phenyl or an indole, which may or may not be substituted, or an aromatic amino acid derivative comprising a phenyl or an indole, which may or may not be substituted.

The present invention relates to a fast-acting insulin formulation comprising at least one substituted anionic compound and at least one polyanionic compound, and said substituted anionic compound per se.

Since the production of insulin by genetic engineering, at the start of the 1980s, diabetic patients have been benefiting from human insulin for their treatment. This product has greatly improved this therapy, since the immunological risks associated with the use of nonhuman insulin, in particular from pigs, are eliminated. However, human insulin injected subcutaneously has a hypoglycemiant effect only after 60 minutes, which means that diabetic patients treated with human insulin must perform the injection 30 minutes before a meal.

One of the problems to solve for improving the health and comfort of diabetic patients is that of providing them with insulin formulations that can provide a faster hypoglycemic response than that of human insulin and, if possible, approaching the physiological response of a healthy person after having taken a meal. The secretion of endogenous insulin in a healthy individual is immediately triggered by the increase in glycemia. The objective is to minimize as far as possible the delay between the injection of insulin and the start of a meal.

It is nowadays accepted that the provision of such formulations is useful in order for the medical care of the patient to be as good as possible.

Genetic engineering has made it possible to provide a response with the development of rapid insulin analogs. These insulins are modified on one or two amino acids so as to be more rapidly absorbed into the blood compartment after a subcutaneous injection. These insulins lispro (Humalog®, Eli Lilly), aspart (Novolog®, Novo Nordisk) and glulisine (Apidra®, Sanofi Aventis) are stable insulin solutions with a faster hypoglycemic response than that of human insulin. Consequently, patients treated with these rapid insulin analogs can perform the insulin injections as little as 15 minutes before a meal.

The principle of rapid insulin analogs is to form hexamers at a concentration of 100 IU/mL to ensure stability of the insulin in the commercial product while at the same time promoting the very rapid dissociation of these hexamers into monomers after subcutaneous injection so as to obtain a rapid action.

Human insulin as formulated in its commercial form does not make it possible to obtain a hypoglycemic response that is close in kinetic terms to the physiological response generated by the start of a meal (increase in glycemia), since, at the working concentration (100 IU/mL), in the presence of zinc and other excipients such as phenol or m-cresol it assembles in the form of hexamers whereas it is active in monomeric and dimeric form. Human insulin is prepared form of hexamers to be stable for close to 2 years at 4° C., since, in the form of monomers, it has a very high propensity to aggregate and then to fibrillate, which causes it to lose its activity. Furthermore, in this aggregated form, it presents an immunological risk for the patient.

Dissociation of the hexamers into dimers and of the dimers into monomers delays its action by up to 20 minutes when compared with a rapid insulin analog (Brange J., et al., Advanced Drug Delivery Review, 35, 1999, 307-335).

In addition, the kinetics of passage of insulin analogs into the blood and the kinetics of glycemia reduction are not optimal, and there is a real need for a formulation that has an even shorter action time so as to approach the secretion kinetics of endogenous insulin in healthy people.

The company Biodel has proposed a solution to this problem with a human insulin formulation comprising EDTA and citric acid, as described in patent application US 2008/39365. By virtue of the capacity of EDTA to complex zinc atoms and by virtue of the interactions of citric acid with the cationic zones present on the surface of insulin, these agents are described as destabilizing the hexameric form of insulin and thus of reducing its action time.

However, such a formulation especially has the drawback of dissociating in the pharmaceutical form the hexameric form of insulin, which is the only stable form that is capable of satisfying the stability requirements of the pharmaceutical regulation.

Patent applications PCT WO 2010/122 385 and WO 2013/064 787, in the name of the Applicant, are also known, which describe formulations of a substituted polysaccharide or oligosaccharide comprising carboxyl groups.

Patent application PCT/FR 2013/052 736 filed on Nov. 13, 2013 in the name of the Applicant, is also known, which describes human insulin or insulin analog formulations and which also makes it possible to solve the various problems mentioned above via the addition of a substituted anionic compound.

The polysaccharides described in patent applications WO 2010/122 385 A1 and US 2012/094 902 A1 as excipients are compounds consisting of chains whose lengths are statistically variable and which have a great richness of sites of possible interaction with protein active principles. This richness might induce a lack of specificity in terms of interaction, and a smaller and better defined molecule might make it possible to be more specific in this subject.

In addition, a molecule with a well-defined backbone is generally more easily traceable (for example MS/MS) in biological media during pharmacokinetic or ADME (administration, distribution, metabolism, elimination) experiments when compared with a polymer which generally gives a very diffuse and noisy signal in mass spectrometry. On the contrary, it is not excluded for a well-defined and shorter molecule to be liable to have a deficit of possible sites of interaction with protein active principles. Specifically, on account of their small size, they do not have the same properties as polymers of polysaccharide type, since there is a loss of the polymer effect.

Despite these potential drawbacks, the Applicant has developed formulations that are capable of accelerating insulin by using a substituted anionic compound in combination with a polyanionic compound.

The present invention, like that described in patent application PCT/FR 2013/052 736, makes it possible to solve the various problems outlined above.

The invention consists of a composition, in aqueous solution, comprising insulin in hexameric form, at least one substituted anionic compound and a polyanionic compound.

The invention consists of a composition, in aqueous solution, comprising insulin in hexameric form, at least one substituted anionic compound and at least one polyanionic compound.
said substituted anionic compound consisting of a discrete number n of between 1 and 8 of identical or different saccharide units, linked via identical or different glycoside bonds, said saccharide unit or one of said saccharide units being in open, oxidized or reduced form, said compound comprising salifiable carboxyl groups and said substituted anionic compound bearing on its reductive chain end at least one radical AA resulting from an aromatic amino acid comprising a phenyl or an indole, which may or may not be substituted, or an aromatic amino acid derivative comprising a phenyl or an indole, which may or may not be substituted.

For a substituted anionic compound, the discrete number n of between 1 and 8 of saccharide units has a single value chosen from the group consisting of 1, 2, 3, 4, 5, 6, 7 and 8.

Said substituted anionic compound, consisting of a discrete number n of between 1 and 8 of identical or different saccharide units, linked via identical or different glycoside bonds, said saccharide unit or one of said saccharide units being in open, oxidized or reduced form, is resulting from a compound consisting of a discrete number n of between 1 and 8 of identical or different saccharide units, linked via identical or different glycoside bonds and bearing at its terminal a reductive chain end.

The term “open saccharide unit” means a saccharide unit resulting from a saccharide unit bearing a reductive terminal.

The term “reductive end group”, “reductive chain end” or “reductive terminal” means the end of the chain formed from a defined number of saccharide units bearing a hemiacetal or aldehyde function. It behaves like a reducing agent in the Tollens test, for example, which makes it possible to assay the chain ends bearing an aldehyde in sugars:

Sugar-CHO+2Ag⁺_{(aq)}+3HO⁻→Sugar-COO⁻+2Ag₍ₛ₎+2H₂O

The term “oxidized form” means that the aldehyde function is in amide form, represented by —C(O)N—.

The term “reduced form” means that the aldehyde function is in amine form, represented by —CH₂—N—.

Said substituted anionic compound consisting of a discrete number n of between 1 and 8 of identical or different saccharide units, linked via identical or different glycoside bonds, comprises a saccharide unit in open, oxidized or reduced form, the n−1 other saccharide units being in closed form, also known as the cyclic form.

According to one embodiment, the radical AA borne by the reductive chain end is linked directly thereto.

According to another embodiment, the radical AA borne by the reductive chain end is linked thereto via a linker arm E, which is at least divalent.

In one embodiment, the linker aim E is resulting from an amino acid, a diamine or an amino alcohol.

According to one embodiment, the substituted anionic compound is chosen from the compounds of formula I, said formula I representing the saccharide unit in open form in which at most one from among R₂, R₃, R₄ and R₆ represents a saccharide backbone formed from a discrete number of closed saccharide units:

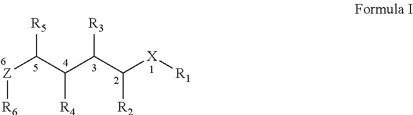

in which
1) Z is either a radical —C═O—, or a radical —CH₂—,
2) X is either a radical —C═O—, or a radical —CH₂—,
3) R₅ is either an —OH radical, or a radical -ƒ-[A]-COOH,
4) R₂, R₃, R₄, R₆, which may be identical or different, are chosen from the group consisting of the radicals —OH, -ƒ-[A]-COOH and at most one from among R₂, R₃, R₄, R₆ is a radical resulting from a saccharide backbone formed from a discrete number n−1 of between 1 and 7 (1≦n−1≦7) of identical or different closed saccharide units, the hydroxyl functions of which may or may not be substituted with a radical -ƒ-[A]-COOH,
5) -[A]- is an at least divalent radical comprising from 1 to 4 carbon atoms chosen from the group consisting of alkyl radicals —(CH₂)ₓ— 1≦x≦4, radicals comprising at least one heteroatom chosen from O, N and S and radicals bearing carboxyl functions and/or -ƒ-[A]-COOH is resulting from an amino acid or an acid alcohol, comprising from 2 to 5 carbon atoms, and is linked to the saccharide units of the compound via a function ƒ;
6) ƒ is chosen from the group consisting of ether, carbamate and amide functions;
7) R₁ is a radical —N(L)ₛ([E]-(o-[AA])ᵤ)ₜ or —N(L)ₛ[AA]
   the linker arm -E- is an at least divalent radical comprising from 1 to 10 carbon atoms optionally comprising at least one heteroatom chosen from O, N and S, and optionally bearing carboxyl functions, and/or —N(L)ₛ-([E]-(o-)ᵤ) is resulting from an amino acid, a diamine, an amino alcohol, an amino diacid, a triamine, a tetramine, an amino diol or an amino triol comprising from 2 to 12 carbon atoms, the amine functions of which are primary and/or secondary;
   -[AA] is resulting from an aromatic amino acid comprising a phenyl or an indole, which may or may not be substituted, or an aromatic amino acid derivative containing a phenyl or an indole, which may or may not be substituted,
   o is an amide, carbamate or carbamide function,
   u=1, 2 or 3 and
   when R₁ is a radical —N(L)ₛ([E]-(o-[AA])ₜ and
      when X is a radical —C═O— then
         S=0 or 1, t=1 or 2 and s+t=2;
         L is chosen from the group consisting of
         —H, and
         a linear or branched alkyl radical comprising from 1 to 4 carbon atoms, and
      when X is a radical —CH₂—, then
         S=0, 1 or 2, t=1 or 2 and s+t=2 or 3;
         if s=1, L is chosen from the group consisting of
         —H, and
         —H and/or -[A]-COOH if ƒ as defined in point 6) is an ether function,
         —H and/or —CO—NH-[A]-COOH if ƒ as defined in point 6) is a carbamate function, and
         a linear or branched alkyl radical comprising from 1 to 4 carbon atoms, and
         if s=2, L is chosen from the group consisting of:
         —H, and
         —H and/or -[A]-COOH if ƒ as defined in point 6) is an ether function, and
         a linear or branched alkyl radical comprising from 1 to 4 carbon atoms, and
      when R₁ is a radical —N(L)ₛ[AA] and
         when X is a radical —C═O— then s=1 and L is chosen from the group consisting of:
         —H,
         a linear or branched alkyl radical comprising from 1 to 4 carbon atoms,
         when X is a radical —CH₂—, then s=1 or 2 and
         if s=1, L is chosen from the group consisting of:
         —H, and
         —H and/or -[A]-COOH if ƒ as defined in point 6) is an ether function,
         —H and/or —CO—NH-[A]-COOH if ƒ as defined in point 6) is a carbamate function, and
         a linear or branched alkyl radical comprising from 1 to 4 carbon atoms, and
         if s=2, L is chosen from the group consisting of
         —H, and
         —H and/or -[A]-COOH if ƒ as defined in point 6) is an ether function, and
         a linear or branched alkyl radical comprising from 1 to 4 carbon atoms, and
8) the degree of substitution represented by p is the number of carboxylate functions per saccharide unit, said carboxylate functions optionally being carboxylate functions that are naturally present on the saccharide units, being resulting from substitution with radicals -[A]-COOH and/or radicals -[AA] and 6≧p≧0.1,
   and the acid functions being in the form of salts of alkali metal cations chosen from the group consisting of Na⁺ and K⁺.

When u is greater than or equal to 2, then the radicals -[AA] may be identical or different.

When t is equal to 2, then -([E]-(o-[AA])ᵤ) may be identical or different.

When s is equal to 2, then:
either t=1 and the nitrogen atom of —N(L)ₛ([E]-(o-[AA])ᵤ) is in quaternary ammonium form, i.e. in the form —N⁺(L)₂([E]-(o-[AA])ᵤ), and
or —N(L)ₛ[AA] is in quaternary ammonium form, i.e. in the form —N⁺(L)₂[AA].

Depending on the pH, the amine functions may or may not be in the form of ammonium salts.

In one embodiment, the substituted anionic compound is chosen from the compounds of formula I in which ƒ is an ether function, and
when R₁ is a radical —N(L)ₛ([E]-(o-[AA])ᵤ)ₜ and N(L)ₛ-([E]-(o-)ᵤ) is resulting from an amino acid, a diamine or an amino alcohol bearing primary amine functions:
when X is a radical —C═O— then
   s=0 or 1, t=1 or 2 and s+t=2 and L is —H, and
when X is a radical —CH₂—, then
   s=0, 1 or 2, t=1 or 2 and s+t=2 or 3 and L is H and/or -[A]-COOH
when R₁ is a radical —N(L)ₛ[AA] and
when X is a radical —C═O— then s=1 and L is —H,
when X is a radical —CH₂—, then s=1 or 2 and L is H and/or -[A]-COOH.

In one embodiment, the substituted anionic compound is chosen from the compounds of formula I in which ƒ is a carbamate function, and
when R₁ is a radical —N(L)ₛ([E]-(o-[AA])ₜ and N(L)ₛ-([E]-(o-)ᵤ) is resulting from an amino acid, a diamine or an amino alcohol bearing primary amine functions:
   when X is a radical —C═O— then
      s=0 or 1, t=1 or 2 and s+t=2 and L is —H, and
   when X is a radical —CH₂—, then
      s=0, 1 or 2, t=1 or 2 and s+t=2 or 3;
         if s=1, L is H and/or —CO—NH-[A]-COOH, and
         if s=2, L is —H, or L is H and —CO—NH-[A]-COOH
   when R₁ is a radical —N(L)ₛ[AA] and
      when X is a radical —C═O— then s=1 and L is —H,
   when X is a radical —CH₂—, then s=1 or 2 and L is H and/or —CO—NH-[A]-COOH.

In one embodiment, the substituted anionic compound is chosen from the compounds of formula I in which, when R₁ is a radical —N(L)ₛ([E]-(o-[AA])ᵤ)ₜ and N(L)ₛ-([E]-(o-)ᵤ) is resulting from an amino acid, a diamine or an amino alcohol bearing secondary amine functions:
when R₁ is a radical —N(L)ₛ([E]-(o-[AA])ᵤ)ₜ and
   when X is a radical —C═O— then
      s=1, t=1 and L is a linear or branched alkyl radical comprising from 1 to 4 carbon atoms, and
   when X is a radical —CH₂—, then
      s=1 or 2, t=1 or 2 and s+t=2 or 3;
      if s=1, L is a linear or branched alkyl radical comprising from 1 to 4 carbon atoms, and
      if s=2, L is chosen from the group consisting of:
         a linear or branched alkyl radical comprising from 1 to 4 carbon atoms, and
         a linear or branched alkyl radical comprising from 1 to 4 carbon atoms, and/or —H, and
         a linear or branched alkyl radical comprising from 1 to 4 carbon atoms, and/or -[A]-COOH if ƒ is an ether function, and
         a linear or branched alkyl radical comprising from 1 to 4 carbon atoms, and/or —CO—NH-[A]-COOH if ƒ is a carbamate function,
when R₁ is a radical —N(L)ₛ[AA] and
   when X is a radical —C═O—, then s=1 and L is a linear or branched alkyl radical comprising from 1 to 4 carbon atoms,
   when X is a radical —CH₂—, then s=1 or 2,
      if s=1, L is a linear or branched alkyl radical comprising from 1 to 4 carbon atoms, and
      if s=2, L is chosen from the group consisting of:
         a linear or branched alkyl radical comprising from 1 to 4 carbon atoms, and
         a linear or branched alkyl radical comprising from 1 to 4 carbon atoms, and/or —H, and
         a linear or branched alkyl radical comprising from 1 to 4 carbon atoms, and/or -[A]-COOH if ƒ is an ether function, and
         a linear or branched alkyl radical comprising from 1 to 4 carbon atoms, and/or —CO—NH-[A]-COOH if ƒ is a carbamate function

The radical -[AA] is resulting from an aromatic amino acid comprising a phenyl or an indole, which may or may not be substituted, or an aromatic amino acid derivative containing a phenyl or an indole, which may or may not be substituted.

The term “aromatic amino acid comprising a substituted or unsubstituted phenyl or indole” means a compound comprising from 7 to 20 carbon atoms, a phenyl or an indole, which may or may not be substituted, at least one amine function and at least one acid function.

According to one embodiment, the substituted anionic compound is chosen from the compounds of formula I in which the radical -[AA] is resulting from an aromatic amino acid comprising a phenyl or an indole, which may or may not be substituted.

The radical -[AA] is linked to the radical -E- or to —X— following a reaction of the amine of the precursor of -[AA], aromatic amino acid or aromatic amino acid derivative, with a precursor of the radical -E- or with the reductive end of the saccharide chain, which is optionally oxidized.

In one embodiment, the substituted anionic compound is chosen from the compounds of formula I in which the radical -[AA] is resulting from an aromatic amino acid comprising a phenyl or an indole, which may or may not be substituted, chosen from alpha- and beta-amino acids.

In one embodiment, the substituted anionic compound is chosen from the compounds of formula I in which the radical -[AA] is resulting from an aromatic amino acid comprising a phenyl or an indole, which may or may not be substituted, comprising only one amine function and only one acid function.

In one embodiment, the substituted anionic compound is chosen from the compounds of formula I in which the radical -[AA] is resulting from an aromatic amino acid comprising a phenyl or an indole, which may or may not be substituted, chosen from the group consisting of phenylalanine, alpha-methylphenylalanine, 3,4-dihydroxyphenylalanine, alpha-phenylglycine, 4-hydroxyphenylglycine, 3,5-phenylglycine, tyrosine, alpha-methyltyrosine, O-methyltyrosine and tryptophan.

In one embodiment, the substituted anionic compound is chosen from the compounds of formula I in which the radical -[AA] is resulting from an aromatic amino acid comprising a phenyl or an indole, which may or may not be substituted, chosen from the group consisting of natural amino acids.

In one embodiment, the natural amino acids are chosen from the group consisting of phenylalanine, tyrosine and tryptophan.

In one embodiment, the natural amino acid is phenylalanine.

The aromatic amino acids comprising a substituted or unsubstituted phenyl or indole, and the derivatives thereof may be in levorotatory or dextrorotatory form or in racemic form.

In one embodiment, it is in levorotatory form.

The term “aromatic amino acid derivative” means decarboxylated derivatives, amino alcohol or amino amide derivatives corresponding to the aromatic amino acids comprising a phenyl or an indole, which may or may not be substituted.

In one embodiment, the “aromatic amino acid derivative” comprising a substituted or unsubstituted phenyl or indole is chosen from the group consisting of amino alcohols and amino amides.

In one embodiment, the substituted anionic compound is chosen from the compounds of formula I in which the function ƒ is an ether function.

In one embodiment, the substituted anionic compound is chosen from the compounds of formula I in which the function f is a carbamate function.

In one embodiment, the substituted anionic compound is chosen from the compounds of formula I in which the function ƒ is an amide function.

In one embodiment, the substituted anionic compound is chosen from the compounds of formula I in which the radical -ƒ-[A]-COOH is chosen from the group consisting of the following sequences, ƒ having the meaning given above:

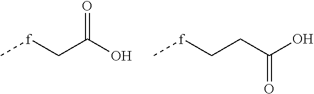

or salts thereof of alkali metal cations chosen from the group consisting of Na⁺ and K⁺.

In one embodiment, the radical -ƒ-[A]-COOH comprises a radical -[A]-comprising 1 or 2 carbon atoms, in particular said radical -[A]- is linked to a saccharide unit via an ether function ƒ.

In one embodiment, the anionic compound is chosen from the compounds of formula I in which the radical -ƒ-[A]-COOH is -ƒ-CH₂—COOH and ƒ is an ether function.

In one embodiment, the substituted anionic compound is chosen from the compounds of formula I in which the radical -ƒ-[A]-COOH is resulting from an amino acid comprising from 2 to 5 carbon atoms; and -ƒ- is an amide or carbamate function.

In one embodiment, ƒ is an amide function.

In one embodiment, the substituted anionic compound is chosen from the compounds of formula I in which the amino acid comprising from 2 to 5 carbon atoms is glycine.

According to one embodiment, the substituted anionic compound comprises at least one carboxylate function. The carboxylate function may be naturally present on the saccharide units, in cyclic or open form, or may originate from a radical -ƒ-[A]-COOH or from a radical -[AA].

According to one embodiment, p≧0.1.

According to one embodiment, p≧0.2.

According to one embodiment, p≧0.3.

According to one embodiment, p≧0.5.

According to one embodiment, p≧0.7.

According to one embodiment, p≧0.9.

According to one embodiment, 3.5≧p.

According to one embodiment, 3.2≧p.

According to one embodiment, 3≧p.

According to one embodiment, 2.8≧p.

According to one embodiment, 2.5≧p.

According to one embodiment, 2≧p.

According to one embodiment, 3.5≧p≧0.1.

According to one embodiment, 3.2≧p≧0.2.

According to one embodiment, 3≧p≧0.3.

According to one embodiment, 2.8≧p≧0.5.

According to one embodiment, 2.5≧p≧0.7.

According to one embodiment, 2≧p≧0.9.

In one embodiment, the substituted anionic compound is chosen from the compounds of formula I in which at most one from among R₂, R₃, R₄ and R₆ is a radical resulting from a backbone formed from a discrete number n−1 of between 1 and 6, i.e. 1≦n−1≦6.

In one embodiment, 1≦n−1≦5.

In one embodiment, 2≦n−1≦4.

According to a second embodiment, n−1 is equal to 1.

According to a second embodiment, n−1 is equal to 2.

According to a second embodiment, n−1 is equal to 3.

According to a second embodiment, n−1 is equal to 4.

According to a second embodiment, n−1 is equal to 5.

According to a second embodiment, n−1 is equal to 6.

According to a second embodiment, n−1 is equal to 7.

The term “backbone” or “saccharide backbone” means a radical formed from a discrete number n−1 between 1 and 7 of identical or different closed saccharide units.

In one embodiment, the substituted anionic compound is chosen from the compounds of formula I in which at most one from among R₂, R₃, R₄ and R₆ is a radical resulting from a backbone formed from a discrete number n−1 of identical or different saccharide units and said saccharide units are chosen from the group consisting of pentoses, hexoses, uronic acids and N-acetylhexosamines.

In one embodiment, at least one saccharide unit of the saccharide backbone is chosen from the group of pentoses.

In one embodiment, the pentoses are chosen from the group consisting of arabinose, ribulose, xylulose, lyxose, ribose, xylose and deoxyribose.

In one embodiment, at least one saccharide unit of the saccharide backbone is chosen from the group of uronic acids.

In one embodiment, the uronic acids are chosen from the group consisting of glucuronic acid, iduronic acid, galacturonic acid, gluconic acid, mucic acid, glucaric acid and galactonic acid.

In one embodiment, at least one saccharide unit of the saccharide backbone is chosen from the group of N-acetylhexosamines.

In one embodiment, the N-acetylhexosamine is chosen from the group consisting of N-acetylgalactosamine, N-acetylglucosamine and N-acetylmannosamine.

In one embodiment, the saccharide units of the saccharide backbone, which may be identical or different, are linked via identical or different glycoside bonds, especially via glycoside bonds of (1,1), (1,2), (1,3), (1,4) and/or (1,6) type.

In one embodiment, the glycoside bonds of the saccharide backbone are of (1,4) or (1,6) type.

In one embodiment, the glycoside bonds of the saccharide backbone are of (1,4) type.

In one embodiment, at least one saccharide unit of the saccharide backbone is chosen from the group of hexoses.

Most particularly, all of the saccharide units of the saccharide backbone are hexoses.

In one embodiment, the hexoses are chosen from the group consisting of mannose, glucose, fructose, sorbose, tagatose, psicose, galactose, allose, altrose, talose, idose, gulose, fucose, fuculose and rhamnose.

In one embodiment, the saccharide backbone is formed from a discrete number n−1=1 saccharide unit chosen from hexoses, more particularly chosen from the group consisting of mannose, glucose, fructose, sorbose, tagatose, psicose, galactose, allose, altrose, talose, idose, gulose, fucose, fuculose and rhamnose.

In one embodiment, the saccharide backbone is formed from a discrete number n−1=2 identical or different saccharide units chosen from hexoses linked via a glycoside bond of (1,1) type.

In one embodiment, the saccharide backbone is formed from a discrete number n−1=2 different saccharide units chosen from hexoses linked via a glycoside bond of (1,1) type, said saccharide backbone being chosen from the group consisting of trehalose and sucrose.

In one embodiment, the saccharide backbone is formed from a discrete number n−1=2 identical or different saccharide units chosen from hexoses linked via a glycoside bond of (1,2) type.

In one embodiment, the saccharide backbone is formed from a discrete number n−1=2 identical or different saccharide units chosen from hexoses linked via a glycoside bond of (1,2) type, said saccharide backbone being kojibiose.

In one embodiment, the saccharide backbone is formed from a discrete number n−1=2 identical or different saccharide units chosen from hexoses linked via a glycoside bond of (1,3) type.

In one embodiment, the saccharide backbone is formed from a discrete number n−1=2 identical or different saccharide units chosen from hexoses linked via a glycoside bond of (1,3) type, said saccharide backbone being nigeriose or laminaribiose.

In one embodiment, the saccharide backbone is formed from a discrete number n−1=2 identical or different saccharide units chosen from hexoses linked via a glycoside bond of (1,4) type.

In one embodiment, the saccharide backbone is formed from a discrete number n−1=2 different saccharide units chosen from hexoses linked via a glycoside bond of (1,4) type, said saccharide backbone being a disaccharide chosen from the group consisting of maltose, lactose and cellobiose.

In one embodiment, the saccharide backbone is formed from a discrete number n−1=2 identical or different saccharide units chosen from hexoses linked via a glycoside bond of (1,6) type.

In one embodiment, the saccharide backbone is formed from a discrete number n−1=2 different saccharide units chosen from hexoses linked via a glycoside bond of (1,6) type, said saccharide backbone being a disaccharide chosen from the group consisting of isomaltose, melibiose and gentiobiose.

In one embodiment, the substituted anionic compound is chosen from anionic compound is consisting of a saccharide backbone formed from a discrete number n−1=2 identical or different saccharide units chosen from hexoses linked via a glycoside bond of (1,6) type, said saccharide backbone being isomaltose.

In one embodiment, the saccharide backbone of the substituted anionic compound is formed from a discrete number 3≦n−1≦8 of identical or different saccharide units.

In one embodiment, at least one of the identical or different saccharide units of which is composed the saccharide backbone formed from a discrete number 3≦n−1≦8 of saccharide units is chosen from the group consisting of hexoses linked via identical or different glycoside bonds.

In one embodiment, the identical or different saccharide units of which is composed the saccharide backbone formed from a discrete number 3≦n−1≦8 of saccharide units are chosen from hexoses and linked via at least one glycoside bond of (1,2) type.

In one embodiment, the identical or different saccharide units of which is composed the saccharide backbone formed from a discrete number 3≦n−1≦8 of saccharide units are chosen from hexoses and linked via at least one glycoside bond of (1,3) type.

In one embodiment, the identical or different saccharide units of which is composed the saccharide backbone formed from a discrete number 3≦n−1≦8 of saccharide units are chosen from hexoses and linked via at least one glycoside bond of (1,4) type.

In one embodiment, the identical or different saccharide units of which is composed the saccharide backbone formed from a discrete number 3≦n−1≦8 of saccharide units are chosen from hexoses and linked via at least one glycoside bond of (1,6) type.

In one embodiment, the saccharide backbone is formed from a discrete number n−1=3 of identical or different saccharide units.

In one embodiment, the three saccharide units of the saccharide backbone are identical.

In one embodiment, two of the three saccharide units of the saccharide backbone are identical.

In one embodiment, the saccharide units of the saccharide backbone are identical or different and are chosen from hexoses, the central hexose being linked to the two other saccharide units via a glycoside bond of (1,2) type and via a glycoside bond of (1,4) type.

In one embodiment, the identical or different saccharide units of the saccharide backbone are chosen from hexoses and the central hexose is linked to the two other saccharide units via a glycoside bond of (1,3) type and via a glycoside bond of (1,4) type.

In one embodiment, the identical or different saccharide units of the saccharide backbone are chosen from hexoses and the central hexose is linked to the two other saccharide units via a glycoside bond of (1,2) type and via a glycoside bond of (1,6) type.

In one embodiment, the identical or different saccharide units of the saccharide backbone are chosen from hexoses and the central hexose is linked to the two other saccharide units via a glycoside bond of (1,2) type and via a glycoside bond of (1,3) type.

In one embodiment, the identical or different saccharide units of the saccharide backbone are chosen from hexoses and the central hexose is linked to the two other saccharide units via a glycoside bond of (1,4) type and via a glycoside bond of (1,6) type.

In one embodiment, the three identical or different saccharide units of the saccharide backbone are hexoses units chosen from the group consisting of mannose and glucose.

In one embodiment, the saccharide backbone is maltotriose.

In one embodiment, the saccharide backbone of the substituted anionic compound is isomaltotriose.

In one embodiment, the saccharide backbone is formed from a discrete number n−1=4 of identical or different saccharide units.

In one embodiment, the four saccharide units of the substituted saccharide backbone are identical.

In one embodiment, three of the four saccharide units of the saccharide backbone are identical.

In one embodiment, the four saccharide units of the saccharide backbone are hexoses units chosen from the group consisting of mannose and glucose.

In one embodiment, the saccharide backbone is maltotetraose.

In one embodiment, the identical or different saccharide units of the saccharide backbone are chosen from hexoses, one terminal hexose is linked to a saccharide unit via a glycoside bond of (1,2) type and the others are linked together via a glycoside bond of (1,6) type.

In one embodiment, the identical or different saccharide units of the saccharide backbone are chosen from hexoses and linked via a glycoside bond of (1,6) type.

In one embodiment, the identical or different saccharide units of the saccharide backbone are chosen from hexoses and linked via a glycoside bond of (1,4) type.

In one embodiment, the saccharide backbone is formed from a discrete number n−1=5 of identical or different saccharide units.

In one embodiment, the five saccharide units of the saccharide backbone are identical.

In one embodiment, the five saccharide units of the saccharide backbone are hexoses units chosen from the group consisting of mannose and glucose.

In one embodiment, the identical or different saccharide units of the saccharide backbone are chosen from hexoses and linked via a glycoside bond of (1,4) type.

In one embodiment, the saccharide backbone is maltopentaose.

In one embodiment, the saccharide backbone of the substituted anionic compound is formed from a discrete number n−1=6 of identical or different saccharide units.

In one embodiment, the six saccharide units of the saccharide backbone are identical. In one embodiment, the identical or different saccharide units of the saccharide backbone are chosen from hexoses and linked via a glycoside bond of (1,4) type.

In one embodiment, the six identical or different saccharide units of the saccharide backbone are hexoses units chosen from the group consisting of mannose and glucose.

In one embodiment, the saccharide backbone is maltohexaose.

In one embodiment, the saccharide backbone is formed from a discrete number n−1=7 of identical or different saccharide units.

In one embodiment, the seven saccharide units of the saccharide backbone are identical.

In one embodiment, the identical or different saccharide units of the saccharide backbone are chosen from hexoses and linked via a glycoside bond of (1,4) type.

In one embodiment, the seven saccharide units of the saccharide backbone are hexoses units chosen from the group consisting of mannose and glucose.

In one embodiment, the saccharide backbone of the substituted anionic compound is maltoheptaose.

The substituted anionic compound is chosen from the compounds of formula I in which the saccharide unit in open form is resulting from a saccharide unit chosen from the group consisting of pentoses, hexoses, uronic acids and N-acetylhexosamines.

In one embodiment, the pentose is chosen from the group consisting of arabinose, ribulose, xylulose, lyxose, ribose, xylose and deoxyribose.

In one embodiment, the hexose is chosen from the group consisting of mannose, glucose, fructose, sorbose, tagatose, psicose, galactose, allose, altrose, talose, idose, gulose, fucose, fuculose and rhamnose.

In one embodiment, the uronic acid is chosen from the group consisting of glucuronic acid, iduronic acid, galacturonic acid, gluconic acid, mucic acid, glucaric acid and galactonic acid.

In one embodiment, the N-acetylhexosamine is chosen from the group consisting of N-acetylgalactosamine, N-acetylglucosamine and N-acetylmannosamine.

In one embodiment, the substituted anionic compound is chosen from the compounds of formula I in which none from among R₂, R₃, R₄ and R₆ is a radical resulting from a saccharide backbone formed from a discrete number n−1 of between 1 and 7 (1≦n−1≦7) identical or different saccharide units. In this embodiment, n=1 and the substituted anionic compound consists of the sole open saccharide unit.

In one embodiment, the substituted anionic compound is chosen from the compounds of formula I in which one from among R₂, R₃, R₄ and R₆ which is a radical resulting from a saccharide backbone is linked to the open saccharide unit via a glycoside bond of (1,2), (1,3), (1,4) or (1,6) type.

In one embodiment, one from among R₂, R₃, R₄ and R₆ which is a radical resulting from a saccharide backbone is linked to the open saccharide unit via a glycoside bond of (1,4) or (1,6) type.

In one embodiment, one from among R₂, R₃, R₄ and R₆ which is a radical resulting from a saccharide backbone is linked to the open saccharide unit via a glycoside bond of (1,4) type.

In one embodiment, the saccharide units of the saccharide backbone and the saccharide unit from which is derived the open saccharide unit are identical.

In one embodiment, the saccharide units of the saccharide backbone and the saccharide unit from which is derived the open saccharide unit are hexoses.

In one embodiment, the saccharide sequence, i.e. the n saccharide unit(s), of the substituted anionic compound is resulting from a natural compound.

In one embodiment, the saccharide sequence of the substituted anionic compound is resulting from a synthetic compound.

In one embodiment, the saccharide sequence of the substituted anionic compound is resulting from a compound obtained by enzymatic degradation of a polysaccharide followed by a purification.

In one embodiment, the saccharide sequence of the substituted anionic compound is resulting from a compound obtained by chemical degradation of a polysaccharide followed by a purification.

In one embodiment, the saccharide sequence of the substituted anionic compound is resulting from a compound obtained via a chemical route, by covalent coupling of precursors of lower molecular weight.

In one embodiment, the saccharide sequence of the substituted anionic compound is resulting from an oligosaccharide chosen from sophorose, lactulose, maltulose, leucrose, rutinose, isomaltulose, fucosyllactose and panose.

In one embodiment, the substituted anionic compound is chosen from a compound bearing a reductive chain end in closed or cyclic form.

The substituted anionic compound comprises at one of its ends an open saccharide unit, in reduced form following a reductive amination reaction (as described, for example, in the publications M. Yalpani et al., Journal of Polymer Science: Polymer Chemistry Edition 1985, 23, 1395-1405, or B. T. Chao et al., Tetrahedron 2005, 61, 5725-5734) or in oxidized form following an oxidation of the hemiacetal function followed by opening of the oxidized ring by reaction with a molecule bearing an amine function (as described, for example, in the publications T. Zhang et al., Macromolecules 1994, 27, 7302-7308 or S. Takeoka et al., Journal of the Chemical Society, Faraday Transactions 1998, 94(15), 2151-2158).

In one embodiment, the radical R₁ is chosen from the radicals of formula —N(L)ₛ-([E]-(o-[AA]ᵤ)ₜ.

In one embodiment, E comprises 1 to 8 carbon atoms.

In one embodiment, E comprises 1 to 6 carbon atoms.

In one embodiment, E comprises 1 to 4 carbon atoms.

In one embodiment, E comprises one or more heteroatoms chosen from O, N and S.

In one embodiment, the radical —N(L)ₛ-([E]-(o-[AA])ₜ is chosen from radicals in which —N(L)ₛ-([E]-(o-)ᵤ)- is an at least divalent radical resulting from an amino acid comprising from 2 to 12 carbon atoms.

In one embodiment, the amino acid comprises from 2 to 10 carbon atoms.

In one embodiment, the amino acid is chosen from the group consisting of glycine, leucine, phenylalanine, lysine, isoleucine, alanine, valine, serine, threonine, aspartic acid and glutamic acid.

In one embodiment, the amino acid is chosen from the group consisting of aspartic acid and glutamic acid.

The amino acid may be either levorotatory or dextrorotatory, or in racemic form.

In one embodiment, the amino acids is levorotatory.

In one embodiment, the radical —N(L)ₛ-([E]-(o-)ᵤ) is an at least divalent radical resulting from a diamine, a triamine, a tetramine, an amino alcohol, an amino diol or an amino triol.

In one embodiment, the amine functions of these compounds are primary amines.

In one embodiment, the amine functions of these compounds are secondary amines bearing a linear or branched alkyl radical comprising from 1 to 4 carbon atoms.

In one embodiment, the radical —N(L)ₛ-([E]-(o-)ᵤ) is an at least divalent radical resulting from a monoethylene or polyethylene glycol amine.

In one embodiment, the radical —N(L)ₛ-([E]-(o-)ᵤ) is an at least divalent radical resulting from a monoethylene or polyethylene glycol amine chosen from the group consisting of ethanolamine, diethylene glycol amine and triethylene glycol amine.

In one embodiment, the radical —N(L)ₛ-([E]-(o-)ᵤ) is an at least divalent radical resulting from a monoethylene or polyethylene glycol diamine.

In one embodiment, the radical —N(L)ₛ-([E]-(o-)ᵤ) is an at least divalent radical resulting from ethylenediamine.

In one embodiment, the radical —N(L)ₛ-([E]-(o-)ᵤ) is an at least divalent radical resulting from a polyethylene glycol diamine chosen from the group consisting of diethylene glycol diamine and triethylene glycol diamine.

According to one embodiment, the radical —N(L)ₛ-([E]-(o-)ᵤ) is a trivalent radical, especially —N(L)ₛ-([E]-(o-)ᵤ) is resulting from a triamine.

In one embodiment, the radical —N(L)ₛ-([E]-(o-)ᵤ) is a trivalent radical, especially —N(L)ₛ-([E]-(o-)ᵤ) is resulting from a triamine, especially —N(L)ₛ-([E]-(o-)ᵤ) is resulting from an amino acid bearing two amine functions, such as lysine or ornithine, amidated with a diamine, such as ethylenediamine.

In one embodiment, the radical —N(L)ₛ-([E]-(o-)ᵤ) is a tetravalent radical, especially —N(L)ₛ-([E]-(o-)ᵤ) is resulting from trishydroxymethylaminomethane, also known as 2-amino-2-hydroxymethyl-1,3-propanediol, or TRIS.

In one embodiment, the substituted anionic compound corresponds to formula I below in which:
1) X is a radical —CH₂—,
2) R₁ is a radical —N(L)ₛ([E]-(o-[AA])ᵤ)ₜ, L, E, AA, s, t, u and o being as defined above.

In one embodiment, L is —H et/or -[A]-COOH if ƒ is an ether function.

In one embodiment, s=1.

In one embodiment, t=1.

In one embodiment, E is resulting from an amino acid, an amino diacid, a diamine, a triamine, an amino alcohol or an amino diol.

In one embodiment, E is resulting from an amino acid, ethylenediamine or ethanolamine.

In one embodiment, -[AA] is resulting from phenylalanine, phenylglycine, tyrosine or tryptophan.

In one embodiment, -[AA] is resulting from phenylalanine.

In one embodiment, u=1 or 2.

In one embodiment, L is —H and/or -[A]-COOH if ƒ is an ether function; s=1; t=1; u=1 or 2; E is resulting from an amino acid, from a diamine, from an amino diacid, from a triamine, from an amino alcohol or from an amino diol; -[AA] is resulting from phenylalanine, phenylglycine, tyrosine or tryptophan.

In one embodiment, L is —H and/or -[A]-COOH if ƒ is an ether function; s=1; t=1; u=1; E is resulting from an amino acid, from a diamine, from an amino diacid, from a triamine, from an amino alcohol or from an amino diol; -[AA] is resulting from phenylalanine, phenylglycine, tyrosine or tryptophan.

In one embodiment, L is —H and/or -[A]-COOH if ƒ is an ether function; s=1; t=1; u=1 or 2; E is resulting from an amino acid, ethylenediamine or ethanolamine; -[AA] is resulting from phenylalanine, phenylglycine, tyrosine or tryptophan.

In one embodiment, L is —H and/or -[A]-COOH if ƒ is an ether function; s=1; t=1; u=1 or 2; E is resulting from an amino acid, from a diamine, from an amino diacid, from a triamine, from an amino alcohol or from an amino diol; -[AA] is resulting from phenylalanine.

In one embodiment, —R₁ is chosen from the radicals of formula —N(L)ₛ([E]-(o-[AA])ᵤ)ₜ in which L, E, AA, s, t, u and o have the meanings given above, and X is a radical —C═O—.

In one embodiment, the substituted anionic compound corresponds to formula I in which:
1) X is a radical —CO—,
2) R₁ is a radical —N(L)ₛ([E]-(o-[AA])ᵤ)ₜ, L, E, AA, s, t, u and o being as defined above.

In one embodiment, L is —H.

In one embodiment, L is a linear or branched alkyl radical comprising from 1 to 4 carbon atoms.

In one embodiment, s=1.

In one embodiment, t=1.

In one embodiment, E is resulting from an amino acid, an amino diacid, a diamine, a triamine, an amino alcohol or an amino diol.

In one embodiment, E is resulting from an amino acid, ethylenediamine or ethanolamine.

In one embodiment, -[AA] is resulting from phenylalanine, phenylglycine, tyrosine or tryptophan.

In one embodiment, -[AA] is resulting from phenylalanine.

In one embodiment, u=1 or 2.

In one embodiment, L is —H; s=1; t=1; u=1 or 2; E is resulting from an amino acid, from a diamine, from an amino diacid, from a triamine, from an amino alcohol or from an amino diol; -[AA] is resulting from phenylalanine, phenylglycine, tyrosine or tryptophan.

In one embodiment, L is —H; s=1; t=1; u=1; E is resulting from an amino acid, from a diamine, from an amino diacid, from a triamine, from an amino alcohol or from an amino diol; -[AA] is resulting from phenylalanine, phenylglycine, tyrosine or tryptophan.

In one embodiment, L is —H; s=1; t=1; u=1 or 2; E is resulting from an amino acid, ethylenediamine or ethanolamine; -[AA] is resulting from phenylalanine, phenylglycine, tyrosine or tryptophan.

In one embodiment, L is —H; s=1; t=1; u=1 or 2; E is resulting from an amino acid, from an amino diacid, from a diamine, from a triamine, from an amino alcohol or from an amino diol; -[AA] is resulting from phenylalanine.

According to one embodiment, the substituted anionic compound corresponds to formula I in which Z is a radical —CH₂— and X is a radical —CH₂— and is chosen from the compounds of formula II:

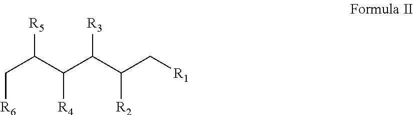

in which:
R₂, R₃, R₄, R₅, R₆, A and p have the values given in the definition of formula I, and
1) ƒ is an ether function;
2) R₁ is a radical —N(L)ₛ[AA], with -[AA] being a radical resulting from an aromatic amino acid or from an aromatic amino acid derivative; s=1 or 2; and
   a. if s=1, L is chosen from the group consisting of —H, —H and/or -[A]-COOH, —H and a linear or branched alkyl radical comprising from 1 to 4 carbon atoms, and
   b. if s=2, L is chosen from —H, —H and/or -[A]-COOH, a linear or branched alkyl radical comprising from 1 to 4 carbon atoms.

According to one embodiment, the substituted anionic compound corresponds to formula I in which Z is a radical —CH₂— and X is a radical —CH₂— and is chosen from the compounds of formula II:

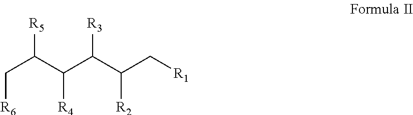

in which:
R₂, R₃, R₄, R₅, R₆, A and p have the values given in the definition of formula I, and
1) ƒ is an ether function, and
2) R₁ is a radical —N(L)ₛ[AA], with -[AA] being a radical resulting from an aromatic amino acid or from an aromatic amino acid derivative; s=1 or 2; and
   a. if s=1, L is chosen from the group consisting of —H, —H and/or -[A]-COOH, and
   b. if s=2, L is chosen from H or —H and/or -[A]-COOH.

According to one embodiment, the substituted anionic compound corresponds to formula I in which Z is a radical —CH₂— and X is a radical —C═O— and is chosen from the compounds of formula III:

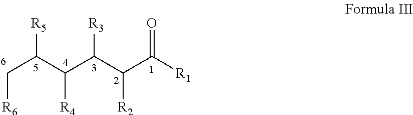

in which
R₂, R₃, R₄, R₅, R₆, A and p have the values given in the definition of formula I, and
1) ƒ is an ether function, and
2) R₁ is a radical —N(L)s[AA], with -[AA] being a radical resulting from an aromatic amino acid or from an aromatic amino acid derivative; s=1; and L is chosen from the group consisting of —H and a linear or branched alkyl radical comprising from 1 to 4 carbon atoms.

According to one embodiment, the substituted anionic compound corresponds to formula I in which Z is a radical —CH₂— and X is a radical —CH₂— and is chosen from the compounds of formula II:

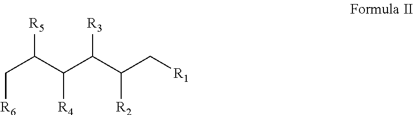

in which:
R₂, R₃, R₄, R₅, R₆, A and p have the values given in the definition of formula I, and
1) ƒ is an ether function, and
2) R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1 or 2; and
   L is chosen from the group consisting of —H, —H and/or -[A]-COOH, —H and a linear or branched alkyl radical comprising from 1 to 4 carbon atoms.

According to one embodiment, the substituted anionic compound corresponds to formula I in which Z is a radical —CH₂— and X is a radical —CH₂— and is chosen from the compounds of formula II:

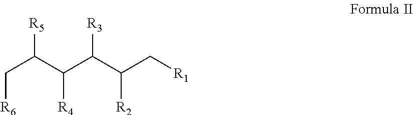

in which:
R₂, R₃, R₄, R₅, R₆, A and p have the values given in the definition of formula I, and
1) ƒ is an ether function, and
2) R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1 or 2; and
   L is chosen from the group consisting of —H, —H and/or -[A]-COOH.

According to one embodiment, the substituted anionic compound corresponds to formula I in which Z is a radical —CH₂— and X is a radical —C(O)— and is chosen from the compounds of formula III:

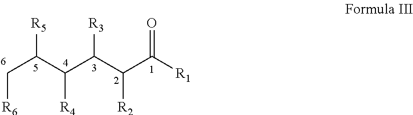

in which
R₂, R₃, R₄, R₅, R₆, A and p have the values given in the definition of formula I, and
1) ƒ is an ether function, and
2) R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1; and L is chosen from —H and a linear or branched alkyl radical comprising from 1 to 4 carbon atoms.

According to one embodiment, the substituted anionic compound corresponds to formula I in which Z is a radical —CH₂— and X is a radical —C═O— and is chosen from the compounds of formula III:

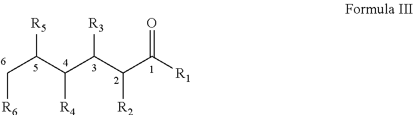

in which
R₂, R₃, R₄, R₅, R₆, A and p have the values given in the definition of formula I, and
1) ƒ is an ether function, and
2) R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1; and L is —H.

Most particularly, in the preceding seven embodiments, the radical -[AA] is resulting from an aromatic amino acid and more particularly from phenylalanine.

According to one embodiment, the substituted anionic compound is chosen from the compounds of formula I in which Z is a radical —CH₂— and R₄ is resulting from a saccharide backbone formed from a discrete number n−1 of glucose saccharide units and is represented by formula IV:

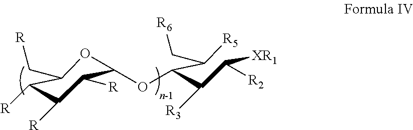

in which
R₁, R₂, R₃, R₅, R₆, X, A and n have the values given in the definition of formula I, and

### R is —OH or -ƒ-[A]-COOH.

According to one embodiment, the substituted anionic compound corresponds to formula IV:

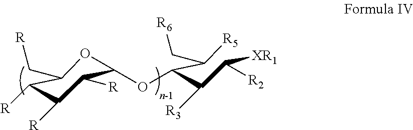

in which
R₂, R₃, R₅, R₆, A and n have the values given in the definition of formula I, and
R is —OH or -ƒ-[A]-COOH, and
X is a radical —CH₂—,
ƒ is an ether function;
R₁ is a radical —N(L)s[AA], with -[AA] being a radical resulting from an aromatic amino acid or an aromatic amino acid derivative; s=1 or 2; and
   a. if s=1, L is chosen from the group consisting of —H, —H and/or -[A]-COOH, —H and a linear or branched alkyl radical comprising from 1 to 4 carbon atoms, and
   b. if s=2, L is chosen from H, —H and/or -[A]-COOH, a linear or branched alkyl radical comprising from 1 to 4 carbon atoms.

According to one embodiment, the substituted anionic compounds correspond to formula IV:

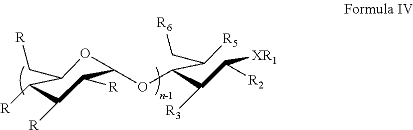

in which
R₂, R₃, R₅, R₆, A and n have the values given in the definition of formula I, and
R is —OH or -ƒ
-[A]-COOH, and
X is a radical —CH₂—,
ƒ is an ether function, and
R₁ is a radical —N(L)ₛ[AA], with -[AA] being a radical resulting from an aromatic amino acid or an aromatic amino acid derivative; s=1 or 2; and
   a. if s=1, L is chosen from the group consisting of —H, —H and/or -[A]-COOH, and
   b. if s=2, L is chosen from H or —H and/or -[A]-COOH.

According to one embodiment, the substituted anionic compound corresponds to formula IV:

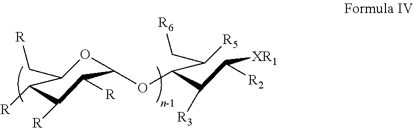

in which
R₂, R₃, R₅, R₆, A and n have the values given in the definition of formula I, and
R is —OH or -ƒ-[A]-COOH, and
X is a radical —C═O—,
ƒ is an ether function, and
R₁ is a radical —N(L)ₛ[AA], with -[AA] being a radical resulting from an aromatic amino acid or from an aromatic amino acid derivative; s=1; and L is chosen from the group consisting of —H and a linear or branched alkyl radical comprising from 1 to 4 carbon atoms.

According to one embodiment, the substituted anionic compound corresponds to formula IV:

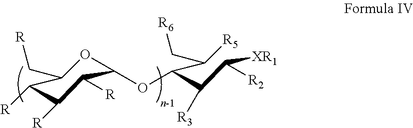

in which
R₂, R₃, R₅ and R₆ have the values given in the definition of formula I, and
R is —OH or -ƒ-[A]-COOH, and
n=3,
-[A]- is a radical —CH₂—,
X is a radical —CH₂—,
ƒ is an ether function, and
R₁ is a radical —N(L)ₛ[AA], with -[AA] being a radical resulting from phenylalanine; s=1 or 2; and L is —H and/or -[A]-COOH.

According to one embodiment, the substituted anionic compound corresponds to formula IV:

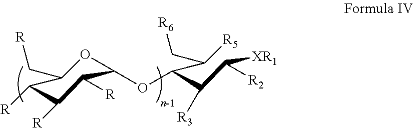

in which
R₂, R₃, R₅, R₆, A and n have the values given in the definition of formula I, and
R is —OH or -ƒ-[A]-COOH, and
X is a radical —CH₂—,
ƒ is an ether function, and
R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1 or 2; and
L is chosen from the group consisting of —H, —H and/or -[A]-COOH and a linear or branched alkyl radical comprising from 1 to 4 carbon atoms.

According to one embodiment, the substituted anionic compound corresponds to formula IV:

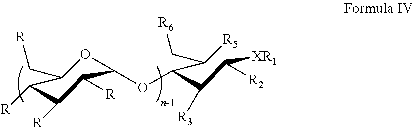

in which
R₂, R₃, R₅, R₆, A and n have the values given in the definition of formula I, and
R is —OH or -ƒ-[A]-COOH, and
X is a radical —CH₂—,
ƒ is an ether function, and
R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1 or 2; and
L is chosen from the group consisting of —H, —H and/or -[A]-COOH.

According to one embodiment, the substituted anionic compound corresponds to formula IV:

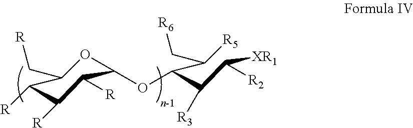

in which
R₂, R₃, R₅, R₆, A and n have the values given in the definition of formula I, and
R is —OH or -ƒ-[A]-COOH, and
X is a radical —C═O—,
ƒ is an ether function, and
R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1; and L is chosen from —H and a linear or branched alkyl radical comprising from 1 to 4 carbon atoms.

According to one embodiment, the substituted anionic compound corresponds to foimula IV:

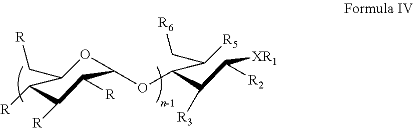

in which
R₂, R₃, R₅, R₆, A and n have the values given in the definition of formula I, and
R is —OH or -ƒ-[A]-COOH, and
X is a radical —C═O—,
ƒ is an ether function, and
R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1; and L is —H.
   Most particularly, in the preceding eight embodiments, the radical -[AA] is resulting from an aromatic amino acid and more particularly from phenylalanine.

The substituted anionic compounds comprise at least one radical -ƒ-[A]-COOH. The radical(s) -ƒ-[A]-COOH may be introduced onto the saccharide units by statistical grafting.

In one embodiment, the substituted anionic compounds are chosen from the substituted anionic compounds in which the radicals -ƒ-[A]-COOH are obtained by grafting at precise positions on the saccharide units via a process involving steps of protection/deprotection of the alcohol or carboxylic acid groups naturally borne by the saccharide units. The strategy leads to selective grafting, especially regioselective grafting, of the substituents onto the saccharide units. The protecting groups include, without limitation, those described in the book (Wuts, P. G. M. et al., Greene's Protective Groups in Organic Synthesis, 2007).

The saccharide precursor of the substituted anionic compound may be obtained by degradation of a high molecular weight polysaccharide. The degradation routes include, without limitation, chemical degradation and/or enzymatic degradation.

The saccharide precursor of the substituted anionic compound may also be obtained by formation of glycoside bonds between monosaccharide or oligosaccharide molecules using a chemical or enzymatic coupling strategy, and the saccharide then obtained comprises a reductive end. The coupling strategies include those described in the publication (Smooth, J. T. et al., Advances in Carbohydrate Chemistry and Biochemistry, 2009, 62, 162-236) and in the book (Lindhorst, T. K., Essentials of Carbohydrate Chemistry and Biochemistry, 2007, 157-209). The coupling reactions may be performed in solution on a solid support. The saccharide molecules before coupling may bear substituents of interest and/or may be functionalized once coupled together statistically or regio selectively.

Thus, by way of example, the substituted anionic compounds may be obtained according to one of the following processes:
the grafting of radicals -ƒ-[A]-COOH by statistical grafting onto the saccharide backbone coupled to the open saccharide unit bearing a radical -[AA]
one or more glycosylation steps between monosaccharide or oligosaccharide molecules bearing radicals -ƒ-[A]-COOH and the saccharide backbone coupled to the open saccharide unit bearing a radical -[AA]
one or more glycosylation steps between one or more monosaccharide or oligosaccharide molecules bearing radicals -ƒ-[A]-COOH and one or more monosaccharide or oligosaccharide molecules and the saccharide backbone coupled to the open saccharide unit bearing a radical -[AA]
one or more steps of introducing protecting groups onto alcohols or acids naturally borne by the saccharide backbone coupled to the open saccharide unit bearing a radical -[AA] followed by one or more grafting reactions to introduce radicals -ƒ-[A]-COOH and finally a step of removing the protecting groups
one or more steps of glycosylation between one or more monosaccharide or oligosaccharide molecules bearing protecting groups on alcohols or acids naturally borne by the saccharide units, one of these saccharides bearing a radical -[AA], one or more grafting steps to introduce radicals -ƒ-[A]-COOH onto the backbone obtained, and then a step of removing the protecting groups,
one or more steps of glycosylation between one or more monosaccharide or oligosaccharide molecules bearing protecting groups on alcohols or acids naturally borne by the saccharide units, and one or more monosaccharide or oligosaccharide molecules, one of these molecules bearing a radical -[AA], one or more grafting steps to introduce -ƒ-[A]-COOH, and then a step of removing the protecting groups,
a step of protecting the reductive chain end present on the precursor of the substituted anionic compound, a step of introducing radicals -ƒ-[A]-COOH onto the saccharide units, a step of deprotecting the reductive chain end and then a step of introducing a radical —N(L)ₛ([E]-(o-[AA])ᵤ)ₜ or —N(L)ₛ[AA] by reaction with the reductive chain end.

The substituted anionic compounds, isolated or as a mixture, may be separated and/or purified in various ways, especially after they have been obtained via the processes described above.

Mention may be made in particular of chromatographic methods, especially “preparative” methods, such as:
flash chromatography, especially on silica, and
chromatographies of the HPLC type (hi-performance liquid chromatography), in particular RP-HPLC (reverse-phase HPLC).

Selective precipitation methods may also be used.

In one embodiment, the mole ratios of substituted anionic compound/insulin are between 0.6 and 75.

In one embodiment, the mole ratios of substituted anionic compound/insulin are between 0.7 and 50.

In one embodiment, the mole ratios of substituted anionic compound/insulin are between 1.4 and 35.

In one embodiment, the mole ratios of substituted anionic compound/insulin are between 1.9 and 30.

In one embodiment, the mole ratios of substituted anionic compound/insulin are between 2.3 and 30.

In one embodiment, the mole ratio of substituted anionic compound/insulin is equal to 8, 12 or 16.

In the above mole ratios, the number of moles of insulin is understood as being the number of moles of insulin monomer.

In one embodiment, the mass ratios of substituted anionic compound/insulin are between 0.5 and 10.

In one embodiment, the mass ratios of substituted anionic compound/insulin are between 0.6 and 7.

In one embodiment, the mass ratios of substituted anionic compound/insulin are between 1.2 and 5.

In one embodiment, the mass ratios of substituted anionic compound/insulin are between 1.6 and 4.

In one embodiment, the mass ratios of substituted anionic compound/insulin are between 2 and 4.

In one embodiment, the mass ratio of substituted anionic compound/insulin is 2, 3, 4 or 6.

In one embodiment, the concentration of substituted anionic compound is between 1.8 and 36 mg/mL.

In one embodiment, the concentration of substituted anionic compound is between 2.1 and 25 mg/mL.

In one embodiment, the concentration of substituted anionic compound is between 4.2 and 18 mg/mL.

In one embodiment, the concentration of substituted anionic compound is between 5.6 and 14 mg/mL.

In one embodiment, the concentration of substituted anionic compound is between 7 and 14 mg/mL.

In one embodiment, the concentration of polyanionic compound is between 5 and 150 mM.

In one embodiment, the concentration of polyanionic compound is between 5 and 100 mM.

In one embodiment, the concentration of polyanionic compound is between 5 and 75 mM.

In one embodiment, the concentration of polyanionic compound is between 5 and 50 mM.

In one embodiment, the concentration of polyanionic compound is between 5 and 30 mM.

In one embodiment, the concentration of polyanionic compound is between 5 and 20 mM.

In one embodiment, the concentration of polyanionic compound is between 5 and 10 mM.

In one embodiment, the concentration of polyanionic compound is between 1 and 30 mg/mL.

In one embodiment, the concentration of polyanionic compound is between 1.5 and 25 mg/mL.

In one embodiment, the concentration of polyanionic compound is between 2 and 25 mg/mL.

In one embodiment, the concentration of polyanionic compound is between 2 and 10 mg/mL.

In one embodiment, the concentration of polyanionic compound is between 2 and 8 mg/mL.

In one embodiment, the insulin is human insulin.

The term “human insulin” means an insulin obtained by synthesis or recombination, the peptide sequence of which is the sequence of human insulin, including the allelic variations and homologs.

In one embodiment, the insulin is a recombinant human insulin as described in the European pharmacopea and the American pharmacopea.

In one embodiment, the insulin is an insulin analog.

The term “insulin analog” means a recombinant insulin whose primary sequence contains at least one modification relative to the primary sequence of human insulin.

In one embodiment, the insulin analog is chosen from the group consisting of the insulin lispro (Humalog®), the insulin aspart (Movolog®, Novorapid®) and the insulin glulisine (Apidra®).

In one embodiment, the insulin analog is the insulin lispro (Humalog®).

In one embodiment, the insulin analog is the insulin aspart (Novolog®, Novorapid®).

In one embodiment, the insulin analog is the insulin glulisine (Apidra®).

In one embodiment, the insulin is in hexameric form.

In one embodiment, the pharmaceutical formulation is characterized in that the insulin concentration is between 240 and 3000 μM (40 to 500 IU/mL).

In one embodiment, the pharmaceutical formulation is characterized in that the insulin concentration is between 600 and 3000 μM (100 to 500 IU/mL).

In one embodiment, the pharmaceutical formulation is characterized in that the insulin concentration is between 600 and 2400 μM (100 to 400 IU/mL).

In one embodiment, the pharmaceutical formulation is characterized in that the insulin concentration is between 600 and 1800 μM (100 to 300 IU/mL).

In one embodiment, the pharmaceutical formulation is characterized in that the insulin concentration is between 600 and 1200 μM (100 to 200 IU/mL).

One embodiment concerns a pharmaceutical formulation characterized in that the insulin concentration is 600 μM (100 IU/mL), 1200 μM (200 IU/mL), 1800 μM (300 IU/mL), 2400 μM (400 IU/mL) or 3000 μM (500 IU/mL).

In one embodiment, the polyanionic compound (PNP) has affinity for zinc lower than the affinity of insulin for zinc and affinity for calcium defined by a dissociation constant Kd_{Ca}=[PNP compound]^{r}[Ca²⁺]^{s}/[(PNP compound)ᵣ-(Ca²⁺)ₛ] is less than or equal to 10^{-1.5}.

This dissociation constant is the reaction constant associated with the dissociation of the complex (PNP compound)ᵣ-(Ca²⁺)ₛ, i.e. with the following reaction: (PNP compound)ᵣ-(Ca²)ₛ≈r(PNP compound)+sCa²⁺.

The dissociation constants (Kd) of the various polyanionic compounds with respect to calcium ions are determined by external calibration using an electrode specific for calcium ions (Mettler Toledo) and a reference electrode. All the measurements are performed in 150 mM of NaCl at pH 7. Only the concentrations of free calcium ions are determined; the calcium ions linked to the polyanionic compound do not induce an electrode potential.

In one embodiment, the polyanionic compound is an anionic molecule chosen from the group consisting of citric acid, aspartic acid, glutamic acid, malic acid, tartaric acid, succinic acid, adipic acid, oxalic acid, phosphate, polyphosphoric acids, such as triphosphate, and the Na⁺, K⁺, Ca²⁺ or Mg²⁺ salts thereof.

In one embodiment, the anionic molecule is citric acid and the Na⁺, K⁺, Ca²⁺ or Mg²⁺ salts thereof.

In one embodiment, the polyanionic compound is chosen from anionic compounds consisting of a saccharide backbone formed from a discrete number u between 1 and 8 (1≦u≦8) of saccharide units, said saccharide units being chosen from the group consisting of hexoses, in cyclic form or in reduced open form, which may be identical or different, linked via identical or different glycoside bonds substituted with carboxyl groups, and salts thereof.

In one embodiment, the polyanionic compound consisting of a saccharide backbone formed from a discrete number of saccharide units is obtained from a disaccharide compound chosen from the group consisting of trehalose, maltose, lactose, sucrose, cellobiose, isomaltose, maltitol and isomaltitol.

In one embodiment, the polyanionic compound consisting of a saccharide backbone formed from a discrete number of saccharide units is obtained from a compound consisting of a backbone formed from a discrete number of saccharide units chosen from the group consisting of maltotriose, maltotetraose, maltopentaose, maltohexaose, maltoheptaose, maltooctaose and isomaltotriose.

In one embodiment, the polyanionic compound consisting of a saccharide backbone formed from a discrete number of saccharide units is chosen from the group consisting of carboxymethylmaltotriose, carboxymethylmaltotetraose, carboxymethylmaltopentaose, carboxymethylmaltohexaose, carboxymethylmaltoheptaose, carboxymethylmaltooactose and carboxymethylisomaltotriose.

In one embodiment, the composition according to the invention comprises insulin, especially as defined above, at least one substituted anionic compound as defined above, and citric acid or the Na⁺, K⁺, Ca²⁺ or Mg²⁺ salts thereof, especially as defined above.

In one embodiment, the composition according to the invention comprises insulin, especially as defined above, at least one substituted anionic compound corresponding to formula I as defined above, and citric acid or the Na⁺, K⁺, Ca²⁺ or Mg²⁺ salts thereof, especially as defined above.

In one embodiment, the composition according to the invention comprises insulin, especially as defined above, at least one substituted anionic compound corresponding to formula II as defined above, and citric acid or the Na⁺, K⁺, Ca²⁺ or Mg²⁺ salts thereof, especially as defined above.

In one embodiment, the composition according to the invention comprises insulin, especially as defined above, at least one substituted anionic compound corresponding to formula III as defined above, and citric acid or the Na⁺, K⁺, Ca²⁺ or Mg²⁺ salts thereof, especially as defined above.

In one embodiment, the composition according to the invention comprises insulin, especially as defined above, at least one substituted anionic compound corresponding to formula IV as defined above, and citric acid or the Na⁺, K⁺, Ca²⁺ or Mg²⁺ salts thereof, especially as defined above.

The invention also relates to the use of a substituted anionic compound of formula I, optionally combined with at least one polyanionic compound, for the preparation of pharmaceutical formulations.

It is known to those skilled in the art that the delay of action of insulins is dependent on the insulin concentration. Only the delay of action values of the formulations at 100 IU/mL are documented.

The “regular” human insulin formulations on the market at a concentration of 600 μM (100 IU/mL) have a delay of action of between 50 and 90 minutes and an end of action of about 360 to 420 minutes in humans. The time to reach the maximum insulin concentration in the blood is between 90 and 180 minutes in humans.

The rapid insulin analog formulations on the market at a concentration of 600 μM (100 IU/mL) have a delay of action of between 30 and 60 minutes and an end of action of about 240-300 minutes in humans. The time to reach the maximum insulin concentration in the blood is between 50 and 90 minutes in humans.

The invention also relates to a method for preparing a human insulin formulation with an insulin concentration of between 240 and 3000 μM (40 and 500 IU/mL), whose delay of action in humans is less than that of the reference formulation at the same insulin concentration in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing a human insulin formulation with an insulin concentration of between 600 and 1200 μM (100 and 200 IU/mL), whose delay of action in humans is less than that of the reference formulation at the same insulin concentration in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing a human insulin formulation with an insulin concentration of 600 μM (100 IU/mL), whose delay of action in humans is less than 60 minutes, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing a human insulin formulation with an insulin concentration of 1200 μM (200 IU/mL), whose delay of action in humans is at least 10% less than that of the human insulin formulation at the same concentration (200 IU/mL) and in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing a human insulin formulation with an insulin concentration of 1800 μM (300 IU/mL), whose delay of action in humans is at least 10% less than that of the human insulin formulation at the same concentration (300 IU/mL) and in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing a human insulin formulation with an insulin concentration of 2400 μM (400 IU/mL), whose delay of action in humans is at least 10% less than that of the human insulin formulation at the same concentration (400 IU/mL) and in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing a human insulin foimulation with an insulin concentration of 3000 μM (500 IU/mL), whose delay of action in humans is at least 10% less than that of the human insulin formulation at the same concentration (500 IU/mL) and in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin is in hexameric form.

The invention consists of the preparation of a “rapid” human insulin formulation, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing a human insulin formulation with an insulin concentration of 600 μM (100 IU/mL), whose delay of action in humans is less than 60 minutes, preferably less than 45 minutes and more preferably less than 30 minutes, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing an insulin analog formulation with an insulin concentration of between 240 and 3000 μM (40 and 500 IU/mL), whose delay of action in humans is less than that of the reference formulation at the same insulin concentration in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin is in hexameric form.

The invention also relates to a method for preparing an insulin analog formulation with an insulin concentration of between 600 and 1200 μM (100 and 200 IU/mL), whose delay of action in man is less than that of the reference formulation at the same insulin analog concentration in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin analog is in hexameric form.

The invention also relates to a method for preparing an insulin analog formulation with an insulin concentration of 600 (100 IU/mL), whose delay of action in humans is less than 30 minutes, characterized in that it comprises (1) a step of adding to said formulation at least one substituted anionic compound, and (2) a step of adding to said formulation at least one polyanionic compound.

In one embodiment, the insulin analog is in hexameric form.

The invention also relates to a method for preparing an insulin analog formulation with an insulin analog concentration of 1200 μM (200 IU/mL), whose delay of action in humans is at least 10% less than that of the insulin analog composition in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin analog is in hexameric form.

The invention also relates to a method for preparing an insulin analog formulation with an insulin analog concentration of 1800 μM (300 IU/mL), whose delay of action in humans is at least 10% less than that of the insulin analog composition in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin analog is in hexameric form.

The invention also relates to a method for preparing an insulin analog formulation with an insulin analog concentration of 2400 μM (400 IU/mL), whose delay of action in humans is at least 10% less than that of the insulin analog composition in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin analog is in hexameric form.

The invention also relates to a method for preparing an insulin analog formulation with an insulin analog concentration of 3000 μM (500 IU/mL), whose delay of action in humans is at least 10% less than that of the insulin analog composition in the absence of substituted anionic compound and of polyanionic compound, characterized in that it comprises (1) a step of adding to said formulation at least one substituted anionic compound, and (2) a step of adding to said formulation at least one polyanionic compound.

In one embodiment, the insulin analog is in hexameric form.

The invention also relates to a pharmaceutical formulation according to the invention, characterized in that it is obtained by drying and/or lyophilization.

In one embodiment, the compositions according to the invention also comprise the addition of zinc salts at a concentration of between 0 and 500 μM, especially between 0 and 300 μM and in particular between 0 and 200 μM.

In one embodiment, the compositions according to the invention comprise buffers at concentrations of between 0 and 100 mM, preferably between 0 and 50 mM or even between 15 and 50 mM.

In one embodiment, the buffer is Tris.

In one embodiment, the compositions according to the invention also comprise preserving agents.

In one embodiment, the preserving agents are chosen from the group consisting of m-cresol and phenol, alone or as a mixture.

In one embodiment, the concentration of preserving agents is between 10 and 50 mM and especially between 10 and 40 mM.

The compositions according to the invention may also comprise additives such as tonicity agents, for instance glycerol, sodium chloride (NaCl), mannitol and glycine.

The compositions according to the invention may also comprise additives in accordance with the pharmacopeas, for instance surfactants, for example polysorbate.

The compositions according to the invention may also comprise any excipient in accordance with the pharmacopeas and compatible with the insulins used at the working concentrations.

In the case of local and systemic releases, the envisaged modes of administration are the intravenous, subcutaneous, intradermal or intramuscular route. Most particularly, the mode of administration is the subcutaneous route.

The transdermal, oral, nasal, vaginal, ocular, buccal and pulmonary administration routes are also envisaged.

The invention also relates to the use of a composition according to the invention for the formulation of a human insulin or insulin analog solution with a concentration of 100 IU/mL, 200 IU/mL or 300 IU/mL intended for implantable or transportable insulin pumps.

According to another of its aspects, the invention also relates to the substituted anionic compounds of formula I as defined below:

In one embodiment, the invention relates to a substituted anionic compound chosen from the compounds of formula I, said formula I representing the saccharide unit in open form in which at most one from among R₂, R₃, R₄ and R₆ represents a saccharide backbone formed from a discrete number of closed saccharide units:

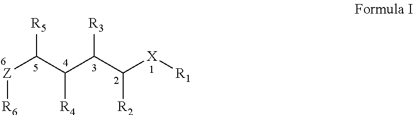

in which
1) Z is either a radical —C═O—, or a radical —CH₂—,
2) X is either a radical —C═O—, or a radical —CH₂—,
3) R₅ is either an —OH radical, or a radical -ƒ-[A]-COOH,
4) R₂, R₃, R₄, R₆, which may be identical or different, are chosen from the group consisting of the radicals —OH, -ƒ-[A]-COOH and at most one from among R₂, R₃, R₄, R₆ is a radical resulting from a saccharide backbone formed from a discrete number n−1 of between 1 and 7 (1≦n−1≦7) of identical or different closed saccharide units, the hydroxyl functions of which may or may not be substituted with a radical -ƒ-[A]-COOH,
5) -[A]- is an at least divalent radical comprising from 1 to 4 carbon atoms chosen from the group consisting of alkyl radicals —(CH₂)ₓ— 1≦x≦4, radicals comprising at least one heteroatom chosen from O, N and S and radicals bearing carboxyl functions and/or -ƒ-[A]-COOH is resulting from an amino acid or an acid alcohol, comprising from 2 to 5 carbon atoms, and is linked to the saccharide units of the compound via a function ƒ;
6) ƒ is chosen from the group consisting of ether, carbamate and amide functions;
7) R₁ is a radical —N(L)ₛ([E]-(o-[AA])ᵤ)ₜ or —N(L)ₛ[AA]
   the linker arm -E- is an at least divalent radical comprising from 1 to 10 carbon atoms optionally comprising at least one heteroatom chosen from O, N and S, and optionally bearing carboxyl functions, and/or —N(L)ₛ-([E]-(ₒ-)u) is resulting from an amino acid, a diamine, an amino alcohol, an amino diacid, a triamine, a tetramine, an amino diol or an amino triol comprising from 2 to 12 carbon atoms, the amine functions of which are primary and/or secondary;
   -[AA] is resulting from an aromatic amino acid comprising a phenyl or an indole, which may or may not be substituted, or an aromatic amino acid derivative containing a phenyl or an indole, which may or may not be substituted,
   o is an amide, carbamate or carbamide function,
   u=1, 2 or 3 and
   when R₁ is a radical —N(L)ₛ([E]-(o-[AA])ᵤ)ₜ and
      when X is a radical —C═O— then
         s=0 or 1, t=1 or 2 and s+t=2;
         L is chosen from the group consisting of
         —H, and
         a linear or branched alkyl radical comprising from 1 to 4 carbon atoms, and
      when X is a radical —CH₂—, then
         s=0, 1 or 2, t=1 or 2 and s+t=2 or 3;
         if s=1, L is chosen from the group consisting of
         —H, and
         —H and/or -[A]-COOH if ƒ as defined in point 6) is an ether function,
         —H and/or —CO—NH-[A]-COOH if ƒ as defined in point 6 is a carbamate function, and
         a linear or branched alkyl radical comprising from 1 to 4 carbon atoms, and
         if s=2, L is chosen from the group consisting of:
         —H, and
         —H and/or -[A]-COOH if ƒ as defined in point 6) is an ether function, and
         a linear or branched alkyl radical comprising from 1 to 4 carbon atoms, and
   when R₁ is a radical —N(L)ₛ[AA] and
      when X is a radical —C═O— then s=1 and L is chosen from the group consisting of:
         —H,
         a linear or branched alkyl radical comprising from 1 to 4 carbon atoms,
      when X is a radical —CH₂—, then s=1 or 2 and
         if s=1, L is chosen from the group consisting of:
         —H, and
         —H and/or -[A]-COOH if ƒ as defined in point 6) is an ether function,
         —H and/or —CO—NH-[A]-COOH if ƒ as defined in point 6) is a carbamate function, and
         a linear or branched alkyl radical comprising from 1 to 4 carbon atoms, and
         if s=2, L is chosen from the group consisting of
         —H, and
         —H and/or -[A]-COOH if ƒ as defined in point 6) is an ether function, and
         a linear or branched alkyl radical comprising from 1 to 4 carbon atoms, and
8) the degree of substitution represented by p is the number of carboxylate functions per saccharide unit, said carboxylate functions optionally being carboxylate functions that are naturally present on the saccharide units, being resulting from substitution with radicals -[A]-COOH and/or radicals -[AA] and 6≧p≧0.1,
   and the acid functions being in the form of salts of alkali metal cations chosen from the group consisting of Na⁺ and K⁺.

When u is greater than or equal to 2, then the radicals -[AA] may be identical or different.

When t is equal to 2, then -([E]-(o-[AA])ᵤ) may be identical or different.

When s is equal to 2, then:
either t=1 and the nitrogen atom of —N(L)ₛ([E]-(o-[AA])ᵤ) is in quaternary ammonium form, i.e. in the form —N⁺(L)₂([E]-(o-[AA])ᵤ), and
or —N(L)ₛ[AA] is in quaternary ammonium form, i.e. in the form —N⁺(L)₂[AA].

In one embodiment, the substituted anionic compound is chosen from the compounds of formula I in which ƒ is an ether function, and
when R₁ is a radical —N(L)ₛ([E]-(o-[AA])ᵤ)ₜ and N(L)ₛ-([E]-(o-)ᵤ) is resulting from an amino acid, a diamine or an amino alcohol bearing primary amine functions:
when X is a radical —C═O— then
   s=0 or 1, t=1 or 2 and s+t=2 and L is —H, and
when X is a radical —CH₂—, then
   s=0, 1 or 2, t=1 or 2 and s+t=2 or 3 and L is H and/or -[A]-COOH
when R₁ is a radical —N(L)ₛ[AA] and
when X is a radical —C═O— then s=1 and L is —H,
when X is a radical —CH₂—, then s=1 or 2 and L is H and/or -[A]-COOH.

In one embodiment, the substituted anionic compound is chosen from the compounds of formula I in which ƒ is a carbamate function, and
when R₁ is a radical —N(L)ₛ([E]-(o-[AA])ₜ and N(L)ₛ-([E]-(o-)ᵤ) is resulting from an amino acid, a diamine or an amino alcohol bearing primary amine functions:
   when X is a radical —C═O— then
      s=0 or 1, t=1 or 2 and s+t=2 and L is —H, and
   when X is a radical —CH₂—, then
      s=0, 1 or 2, t=1 or 2 and s+t=2 or 3;
         if s=1, L is H and/or —CO—NH-[A]-COOH, and
         if s=2, L is —H, or L is H and —CO—NH-[A]-COOH
when R₁ is a radical —N(L)ₛ[AA] and
   when X is a radical —C═O— then s=1 and L is —H,
   when X is a radical —CH₂—, then s=1 or 2 and L is H and/or —CO—NH-[A]-COOH.

In one embodiment, the substituted anionic compound is chosen from the compounds of formula I in which, when R₁ is a radical —N(L)ₛ([E]-(o-[AA])ᵤ)ₜ and N(L)ₛ-([E]-(o-)ᵤ) is resulting from an amino acid, a diamine or an amino alcohol bearing secondary amine functions:
when R₁ is a radical —N(L)ₛ([E]-(o-[AA])ᵤ)ₜ and
   when X is a radical —C═O— then
      s=1, t=1 and L is a linear or branched alkyl radical comprising from 1 to 4 carbon atoms, and
   when X is a radical —CH₂—, then
      s=1 or 2, t=1 or 2 and s+t=2 or 3;
      if s=1, L is a linear or branched alkyl radical comprising from 1 to 4 carbon atoms, and
      if s=2, L is chosen from the group consisting of:
         a linear or branched alkyl radical comprising from 1 to 4 carbon atoms, and
         a linear or branched alkyl radical comprising from 1 to 4 carbon atoms, and/or —H, and
         a linear or branched alkyl radical comprising from 1 to 4 carbon atoms, and/or -[A]-COOH if ƒ is an ether function, and
         a linear or branched alkyl radical comprising from 1 to 4 carbon atoms, and/or —CO—NH-[A]-COOH if ƒ is a carbamate function,
when R₁ is a radical —N(L)ₛ[AA] and
   when X is a radical —C═O—, then s=1 and L is a linear or branched alkyl radical comprising from 1 to 4 carbon atoms,
   when X is a radical —CH₂—, then s=1 or 2,
      if s=1, L is a linear or branched alkyl radical comprising from 1 to 4 carbon atoms, and
      if s=2, L is chosen from the group consisting of:
         a linear or branched alkyl radical comprising from 1 to 4 carbon atoms, and
         a linear or branched alkyl radical comprising from 1 to 4 carbon atoms, and/or —H, and
         a linear or branched alkyl radical comprising from 1 to 4 carbon atoms, and/or -[A]-COOH if ƒ is an ether function, and
         a linear or branched alkyl radical comprising from 1 to 4 carbon atoms, and/or —CO—NH-[A]-COOH if ƒ is a carbamate function.

The radical -[AA] is resulting from an aromatic amino acid comprising a phenyl or an indole, which may or may not be substituted, or an aromatic amino acid derivative containing a phenyl or an indole, which may or may not be substituted.

The term “aromatic amino acid comprising a substituted or unsubstituted phenyl or indole” means a compound comprising from 7 to 20 carbon atoms, a phenyl or an indole, which may or may not be substituted, at least one amine function and at least one acid function.

According to one embodiment, the substituted anionic compound is chosen from the compounds of formula I in which the radical -[AA] is resulting from an aromatic amino acid comprising a phenyl or an indole, which may or may not be substituted.

The radical -[AA] is linked to the radical -E- or to —X— following a reaction of the amine of the precursor of -[AA], aromatic amino acid or aromatic amino acid derivative, with a precursor of the radical -E- or with the reductive end of the saccharide chain, which is optionally oxidized.

In one embodiment, the substituted anionic compound is chosen from the compounds of formula I in which the radical -[AA] is resulting from an aromatic amino acid comprising a phenyl or an indole, which may or may not be substituted, chosen from alpha- and beta-amino acids.

In one embodiment, the substituted anionic compound is chosen from the compounds of formula I in which the radical -[AA] is resulting from an aromatic amino acid comprising a phenyl or an indole, which may or may not be substituted, comprising only one amine function and only one acid function.

In one embodiment, the substituted anionic compound is chosen from the compounds of formula I in which the radical -[AA] is resulting from an aromatic amino acid comprising a phenyl or an indole, which may or may not be substituted, chosen from the group consisting of phenylalanine, alpha-methylphenylalanine, 3,4-dihydroxyphenylalanine, alpha-phenylglycine, 4-hydroxyphenylglycine, 3,5-phenylglycine, tyrosine, alpha-methyltyrosine, O-methyltyrosine and tryptophan.

In one embodiment, the substituted anionic compound is chosen from the compounds of formula I in which the radical -[AA] is resulting from an aromatic amino acid comprising a phenyl or an indole, which may or may not be substituted, chosen from the group consisting of natural amino acids.

In one embodiment, the natural amino acids are chosen from the group consisting of phenylalanine, tyrosine and tryptophan.

In one embodiment, the natural amino acids is phenylalanine.

The aromatic amino acids comprising a substituted or unsubstituted phenyl or indole, and the derivatives thereof may be in levorotatory or dextrorotatory form or in racemic form.

In one embodiment, it is in levorotatory four′.

The term “aromatic amino acid derivative” means decarboxylated derivatives, amino alcohol or amino amide derivatives corresponding to the aromatic amino acids comprising a phenyl or an indole, which may or may not be substituted.

In one embodiment, the “aromatic amino acid derivative” comprising a substituted or unsubstituted phenyl or indole is chosen from the group consisting of amino alcohols and amino amides.

In one embodiment, the substituted anionic compound is chosen from the compounds of formula I in which the function -ƒ is an ether function.

In one embodiment, the substituted anionic compound is chosen from the compounds of formula I in which the function ƒ is a carbamate function.

In one embodiment, the substituted anionic compound is chosen from the compounds of formula I in which the function ƒ is an amide function.

In one embodiment, the substituted anionic compound is chosen from the compounds of formula I in which the radical -ƒ-[A]-COOH is chosen from the group consisting of the following sequences, ƒ having the meaning given above:

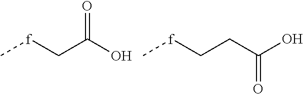

or salts thereof of alkali metal cations chosen from the group consisting of Na⁺ and K⁺.

In one embodiment, the radical -ƒ-[A]-COOH comprises a radical -[A]-comprising 1 or 2 carbon atoms, in particular said radical -[A]- is linked to a saccharide unit via an ether function ƒ.

In one embodiment, the anionic compound is chosen from the compounds of formula I in which the radical -ƒ-[A]-COOH is -ƒ-CH₂—COOH and ƒ is an ether function.

In one embodiment, the substituted anionic compound is chosen from the compounds of formula I in which the radical -ƒ-[A]-COOH is resulting from an amino acid comprising from 2 to 5 carbon atoms; and -ƒ- is an amide or carbamate function.

In one embodiment, ƒ is an amide function.

In one embodiment, the substituted anionic compound is chosen from the compounds of formula I in which the amino acid comprising from 2 to 5 carbon atoms is glycine.

According to one embodiment, the substituted anionic compound comprises at least one carboxylate function. The carboxylate function may be natural the present on the saccharide units, in cyclic or open form, or may originate from a radical -ƒ-[A]-COOH or from a radical -[AA].

According to one embodiment, p≧0.1.

According to one embodiment, p≧0.2.

According to one embodiment, p≧0.3.

According to one embodiment, p≧0.5.

According to one embodiment, p≧0.7.

According to one embodiment, p≧0.9.

According to one embodiment, 3.5≧p.

According to one embodiment, 3.2≧p.

According to one embodiment, 3≧p.

According to one embodiment, 2.8≧p.

According to one embodiment, 2.5≧p.

According to one embodiment, 2≧p.

According to one embodiment, 3.5≧p≧0.1.

According to one embodiment, 3.2≧p≧0.2.

According to one embodiment, 3≧p≧0.3.

According to one embodiment, 2.8≧p≧0.5.

According to one embodiment, 2.5≧p≧0.7.

According to one embodiment, 2≧p≧0.9.

In one embodiment, the substituted anionic compound is chosen from the compounds of formula I in which at most one from among R₂, R₃, R₄ and R₆ is a radical resulting from a backbone formed from a discrete number n−1 of between 1 and 6, i.e. 1≦n−1≦6.

In one embodiment, 1≦n−1≦5.

In one embodiment, 2≦n−1≦4.

According to a second embodiment, n−1 is equal to 1.

According to a second embodiment, n−1 is equal to 2.

According to a second embodiment, n−1 is equal to 3.

According to a second embodiment, n−1 is equal to 4.

According to a second embodiment, n−1 is equal to 5.

According to a second embodiment, n−1 is equal to 6.

According to a second embodiment, n−1 is equal to 7.

In one embodiment, the substituted anionic compound is chosen from the compounds of formula I in which at most one from among R₂, R₃, R₄ and R₆ is a radical resulting from a backbone formed from a discrete number n−1 of identical or different saccharide units and said saccharide units are chosen from the group consisting of pentoses, hexoses, uronic acids and N-acetylhexosamines.

In one embodiment, at least one saccharide unit of the saccharide backbone is chosen from the group of pentoses.

In one embodiment, the pentoses are chosen from the group consisting of arabinose, ribulose, xylulose, lyxose, ribose, xylose and deoxyribose.

In one embodiment, at least one saccharide unit of the saccharide backbone is chosen from the group of uronic acids.

In one embodiment, the uronic acids are chosen from the group consisting of glucuronic acid, iduronic acid, galacturonic acid, gluconic acid, mucic acid, glucaric acid and galactonic acid.

In one embodiment, at least one saccharide unit of the saccharide backbone is chosen from the group of N-acetylhexosamines.

In one embodiment, the N-acetylhexosamine is chosen from the group consisting of N-acetylgalactosamine, N-acetylglucosamine and N-acetylmannosamine.

In one embodiment, the saccharide units of the saccharide backbone, which may be identical or different, are linked via identical or different glycoside bonds, especially via glycoside bonds of (1,1), (1,2), (1,3), (1,4) and/or (1,6) type.

In one embodiment, the glycoside bonds of the saccharide backbone are of (1,4) or (1,6) type.

In one embodiment, the glycoside bonds of the saccharide backbone are of (1,4) type.

In one embodiment, at least one saccharide unit of the saccharide backbone is chosen from the group of hexoses.

Most particularly, all of the saccharide units of the saccharide backbone are hexoses.

In one embodiment, the hexoses are chosen from the group consisting of mannose, glucose, fructose, sorbose, tagatose, psicose, galactose, allose, altrose, talose, idose, gulose, fucose, fuculose and rhamnose.

In one embodiment, the saccharide backbone is formed from a discrete number n−1=1 saccharide unit chosen from hexoses, more particularly chosen from the group consisting of mannose, glucose, fructose, sorbose, tagatose, psicose, galactose, allose, altrose, talose, idose, gulose, fucose, fuculose and rhamnose.

In one embodiment, the saccharide backbone is formed from a discrete number n−1=2 identical or different saccharide units chosen from hexoses linked via a glycoside bond of (1,1) type.

In one embodiment, the saccharide backbone is formed from a discrete number n−1=2 different saccharide units chosen from hexoses linked via a glycoside bond of (1,1) type, said saccharide backbone being chosen from the group consisting of trehalose and sucrose.

In one embodiment, the saccharide backbone is formed from a discrete number n−1=2 identical or different saccharide units chosen from hexoses linked via a glycoside bond of (1,2) type.

In one embodiment, the saccharide backbone is formed from a discrete number n−1=2 identical or different saccharide units chosen from hexoses linked via a glycoside bond of (1,2) type, said saccharide backbone being kojibiose.

In one embodiment, the saccharide backbone is formed from a discrete number n−1=2 identical or different saccharide units chosen from hexoses linked via a glycoside bond of (1,3) type.

In one embodiment, the saccharide backbone is formed from a discrete number n−1=2 identical or different saccharide units chosen from hexoses linked via a glycoside bond of (1,3) type, said saccharide backbone being nigeriose or laminaribiose.

In one embodiment, the saccharide backbone is formed from a discrete number n−1=2 identical or different saccharide units chosen from hexoses linked via a glycoside bond of (1,4) type.

In one embodiment, the saccharide backbone is formed from a discrete number n−1=2 identical or different saccharide units chosen from hexoses linked via a glycoside bond of (1,4) type, said saccharide backbone being a disaccharide chosen from the group consisting of maltose, lactose and cellobiose.

In one embodiment, the saccharide backbone is formed from a discrete number n−1=2 identical or different saccharide units chosen from hexoses linked via a glycoside bond of (1,6) type.

In one embodiment, the saccharide backbone is formed from a discrete number n−1=2 different saccharide units chosen from hexoses linked via a glycoside bond of (1,6) type, said saccharide backbone being a disaccharide chosen from the group consisting of isomaltose, melibiose and gentiobiose.

In one embodiment, the substituted anionic compound is chosen from anionic compound is consisting of a saccharide backbone formed from a discrete number n−1=2 identical or different saccharide units chosen from hexoses linked via a glycoside bond of (1,6) type, said saccharide backbone being isomaltose.

In one embodiment, the saccharide backbone of the substituted anionic compound is formed from a discrete number 3≦n−1≦8 of identical or different saccharide units.

In one embodiment, at least one of the identical or different saccharide units of which is composed the saccharide backbone formed from a discrete number 3≦n−1≦8 of saccharide units is chosen from the group consisting of hexoses linked via identical or different glycoside bonds.

In one embodiment, the identical or different saccharide units of which is composed the saccharide backbone formed from a discrete number 3≦n−1≦8 of saccharide units are chosen from hexoses and linked via at least one glycoside bond of (1,2) type.

In one embodiment, the identical or different saccharide units of which is composed the saccharide backbone formed from a discrete number 3≦n−1≦8 of saccharide units are chosen from hexoses and linked via at least one glycoside bond of (1,3) type.

In one embodiment, the identical or different saccharide units of which is composed the saccharide backbone formed from a discrete number 3≦n−1≦8 of saccharide units are chosen from hexoses and linked via at least one glycoside bond of (1,4) type.

In one embodiment, the identical or different saccharide units of which is composed the saccharide backbone formed from a discrete number 3≦n−1≦8 of saccharide units are chosen from hexoses and linked via at least one glycoside bond of (1,6) type.

In one embodiment, the saccharide backbone is formed from a discrete number n−1-3 of identical or different saccharide units.

In one embodiment, the three saccharide units of the saccharide backbone are identical.

In one embodiment, two of the three saccharide units of the saccharide backbone are identical.

In one embodiment, the saccharide units of the saccharide backbone are identical or different and are chosen from hexoses, the central hexose being linked to the two other saccharide units via a glycoside bond of (1,2) type and via a glycoside bond of (1,4) type.

In one embodiment, the identical or different saccharide units of the saccharide backbone are chosen from hexoses and the central hexose is linked to the two other saccharide units via a glycoside bond of (1,3) type and via a glycoside bond of (1,4) type.

In one embodiment, the identical or different saccharide units of the saccharide backbone are chosen from hexoses and the central hexose is linked to the two other saccharide units via a glycoside bond of (1,2) type and via a glycoside bond of (1,6) type.

In one embodiment, the identical or different saccharide units of the saccharide backbone are chosen from hexoses and the central hexose is linked to the two other saccharide units via a glycoside bond of (1,2) type and via a glycoside bond of (1,3) type.

In one embodiment, the identical or different saccharide units of the saccharide backbone are chosen from hexoses and the central hexose is linked to the two other saccharide units via a glycoside bond of (1,4) type and via a glycoside bond of (1,6) type.

In one embodiment, the three identical or different saccharide units of the saccharide backbone are hexoses units chosen from the group consisting of mannose and glucose.

In one embodiment, the saccharide backbone is maltotriose.

In one embodiment, the saccharide backbone of the substituted anionic compound is isomaltotriose.

In one embodiment, the saccharide backbone is formed from a discrete number n−1=4 of identical or different saccharide units.

In one embodiment, the four saccharide units of the substituted saccharide backbone are identical.

In one embodiment, three of the four saccharide units of the saccharide backbone are identical.

In one embodiment, the four saccharide units of the saccharide backbone are hexoses units chosen from the group consisting of mannose and glucose.

In one embodiment, the saccharide backbone is maltotetraose.

In one embodiment, the identical or different saccharide units of the saccharide backbone are chosen from hexoses, one terminal hexose is linked to a saccharide unit via a glycoside bond of (1,2) type and the others are linked together via a glycoside bond of (1,6) type.

In one embodiment, the identical or different saccharide units of the saccharide backbone are chosen from hexoses and linked via a glycoside bond of (1,6) type.

In one embodiment, the identical or different saccharide units of the saccharide backbone are chosen from hexoses and linked via a glycoside bond of (1,4) type.

In one embodiment, the saccharide backbone is formed from a discrete number n−1=5 of identical or different saccharide units.

In one embodiment, the five saccharide units of the saccharide backbone are identical.

In one embodiment, the five saccharide units of the saccharide backbone are hexoses units chosen from the group consisting of mannose and glucose.

In one embodiment, the identical or different saccharide units of the saccharide backbone are chosen from hexoses and linked via a glycoside bond of (1,4) type.

In one embodiment, the saccharide backbone is maltopentaose.

In one embodiment, the saccharide backbone of the substituted anionic compound is formed from a discrete number n−1=6 of identical or different saccharide units.

In one embodiment, the six saccharide units of the saccharide backbone are identical. In one embodiment, the identical or different saccharide units of the saccharide backbone are chosen from hexoses and linked via a glycoside bond of (1,4) type.

In one embodiment, the six identical or different saccharide units of the saccharide backbone are hexoses units chosen from the group consisting of mannose and glucose.

In one embodiment, the saccharide backbone is maltohexaose.

In one embodiment, the saccharide backbone is formed from a discrete number n−1=7 of identical or different saccharide units.

In one embodiment, the seven saccharide units of the saccharide backbone are identical.

In one embodiment, the identical or different saccharide units of the saccharide backbone are chosen from hexoses and linked via a glycoside bond of (1,4) type.

In one embodiment, the seven saccharide units of the saccharide backbone are hexoses units chosen from the group consisting of mannose and glucose.

In one embodiment, the saccharide backbone of the substituted anionic compound is maltoheptaose.

The substituted anionic compound is chosen from the compounds of formula I in which the saccharide unit in open form is resulting from a saccharide unit chosen from the group consisting of pentoses, hexoses, uronic acids and N-acetylhexosamines.

In one embodiment, the pentose is chosen from the group consisting of arabinose, ribulose, xylulose, lyxose, ribose, xylose and deoxyribose.

In one embodiment, the hexose is chosen from the group consisting of mannose, glucose, fructose, sorbose, tagatose, psicose, galactose, allose, altrose, talose, idose, gulose, fucose, fuculose and rhamnose.

In one embodiment, the uronic acid is chosen from the group consisting of glucuronic acid, iduronic acid, galacturonic acid, gluconic acid, mucic acid, glucaric acid and galactonic acid.

In one embodiment, the N-acetylhexosamine is chosen from the group consisting of N-acetylgalactosamine, N-acetylglucosamine and N-acetylmannosamine.

In one embodiment, the substituted anionic compound is chosen from the compounds of formula I in which none from among R₂, R₃, R₄ and R₆ is a radical resulting from a saccharide backbone formed from a discrete number n−1 of between 1 and 7 (1≦n−1≦7) identical or different saccharide units. In this embodiment, n=1 and the substituted anionic compound consists of the sole open saccharide unit.

In one embodiment, the substituted anionic compound is chosen from the compounds of formula I in which one from among R₂, R₃, R₄ and R₆ which is a radical resulting from a saccharide backbone is linked to the open saccharide unit via a glycoside bond of (1,2), (1,3), (1,4) or (1,6) type.

In one embodiment, one from among R₂, R₃, R₄ and R₆ which is a radical resulting from a saccharide backbone is linked to the open saccharide unit via a glycoside bond of (1,4) or (1,6) type.

In one embodiment, one from among R₂, R₃, R₄ and R₆ which is a radical resulting from a saccharide backbone is linked to the open saccharide unit via a glycoside bond of (1,4) type.

In one embodiment, the saccharide units of the saccharide backbone and the saccharide unit from which is derived the open saccharide unit are identical.

In one embodiment, the saccharide units of the saccharide backbone and the saccharide unit from which is derived the open saccharide unit are hexoses.

In one embodiment, the saccharide sequence, i.e. the n saccharide unit(s), of the substituted anionic compound is resulting from a natural compound.

In one embodiment, the saccharide sequence of the substituted anionic compound is resulting from a synthetic compound.

In one embodiment, the saccharide sequence of the substituted anionic compound is resulting from a compound obtained by enzymatic degradation of a polysaccharide followed by a purification.

In one embodiment, the saccharide sequence of the substituted anionic compound is resulting from a compound obtained by chemical degradation of a polysaccharide followed by a purification.

In one embodiment, the saccharide sequence of the substituted anionic compound is resulting from a compound obtained via a chemical route, by covalent coupling of precursors of lower molecular weight.

In one embodiment, the saccharide sequence of the substituted anionic compound is resulting from an oligosaccharide chosen from sophorose, lactulose, maltulose, leucrose, rutinose, isomaltulose, fucosyllactose and panose.

In one embodiment, the substituted anionic compound is chosen from a compound bearing a reductive chain end in closed or cyclic form.

The substituted anionic compound comprises at one of its ends an open saccharide unit, in reduced form following a reductive amination reaction (as described, for example, in the publications M. Yalpani et al., Journal of Polymer Science: Polymer Chemistry Edition 1985, 23, 1395-1405, or B. T. Chao et al., Tetrahedron 2005, 61, 5725-5734) or in oxidized form following an oxidation of the hemiacetal function followed by opening of the oxidized ring by reaction with a molecule bearing an amine function (as described, for example, in the publications T. Zhang et al., Macromolecules 1994, 27, 7302-7308 or S. Takeoka et al., Journal of the Chemical Society, Faraday Transactions 1998, 94(15), 2151-2158).

In one embodiment, the radical R₁ is chosen from the radicals of formula —N(L)ₛ-([E]-(o-[AA]ᵤ)ₜ.

In one embodiment, E comprises 1 to 8 carbon atoms.

In one embodiment, E comprises 1 to 6 carbon atoms.

In one embodiment, E comprises 1 to 4 carbon atoms.

In one embodiment, E comprises one or more heteroatoms chosen from O, N and S.

In one embodiment, the radical —N(L)ₛ-([E]-(o-[AA]ᵤ)ₜ is chosen from radicals in which —N(L)ₛ-([E]-(o-)ₜ)- is an at least divalent radical resulting from an amino acid comprising from 2 to 12 carbon atoms.

In one embodiment, the amino acid comprises from 2 to 10 carbon atoms.

In one embodiment, the amino acid is chosen from the group consisting of glycine, leucine, phenylalanine, lysine, isoleucine, alanine, valine, serine, threonine, aspartic acid and glutamic acid.

In one embodiment, the amino acid is chosen from the group consisting of aspartic acid and glutamic acid.

The amino acid may be either levorotatory or dextrorotatory, or in racemic form.

In one embodiment, the amino acids is levorotatory.

In one embodiment, the radical —N(L)ₛ-([E]-(o-)ᵤ) is an at least divalent radical resulting from a diamine, a triamine, a tetramine, an amino alcohol, an amino diol or an amino triol.

In one embodiment, the amine functions of these compounds are primary amines.

In one embodiment, the amine functions of these compounds are secondary amines bearing a linear or branched alkyl radical comprising from 1 to 4 carbon atoms.

In one embodiment, the radical —N(L)ₛ-([E]-(o-)ᵤ) is an at least divalent radical resulting from a monoethylene or polyethylene glycol amine.

In one embodiment, the radical —N(L)ₛ-([E]-(o-)ᵤ) is an at least divalent radical resulting from a monoethylene or polyethylene glycol amine chosen from the group consisting of ethanolamine, diethylene glycol amine and triethylene glycol amine.

In one embodiment, the radical —N(L)ₛ-([E]-(o-)ᵤ) is an at least divalent radical resulting from a monoethylene or polyethylene glycol diamine.

In one embodiment, the radical —N(L)ₛ-([E]-(o-)ᵤ) is an at least divalent radical resulting from ethylenediamine.

In one embodiment, the radical —N(L)ₛ-([E]-(o-)ᵤ) is an at least divalent radical resulting from a polyethylene glycol diamine chosen from the group consisting of diethylene glycol diamine and triethylene glycol diamine.

According to one embodiment, the radical —N(L)ₛ-([E]-(o-)ᵤ) is a trivalent radical, especially —N(L)ₛ-([E]-(o-)ᵤ) is resulting from a triamine.

In one embodiment, the radical —N(L)ₛ-([E]-(o-)ᵤ) is a trivalent radical, especially —N(L)ₛ-([E]-(o-)ᵤ) is resulting from a triamine, especially —N(L)ₛ([E]-(o-)ᵤ) is resulting from an amino acid bearing two amine functions, such as lysine or ornithine, amidated with a diamine, such as ethylenediamine.

In one embodiment, the radical —N(L)ₛ-([E]-(o-)ᵤ) is a tetravalent radical, especially —N(L)ₛ-([E]-(o-)ᵤ) is resulting from trishydroxymethylaminomethane, also known as 2-amino-2-hydroxymethyl-1,3-propanediol, or TRIS.

In one embodiment, the substituted anionic compound corresponds to formula I in which:
3) X is a radical —CH₂—,
4) R₁ is a radical —N(L)ₛ([E]-(o-[AA])ᵤ)ₜ, L, E, AA, s, t, u and o being as defined above.

In one embodiment, L is —H et/or -[A]-COOH if ƒ is an ether function.

In one embodiment, s=1.

In one embodiment, t=1.

In one embodiment, E is resulting from an amino acid, an amino diacid, a diamine, a triamine, an amino alcohol or an amino diol.

In one embodiment, E is resulting from an amino acid, ethylenediamine or ethanolamine.

In one embodiment, -[AA] is resulting from phenylalanine, phenylglycine, tyrosine or tryptophan.

In one embodiment, -[AA] is resulting from phenylalanine.

In one embodiment, u=1 or 2.

In one embodiment, L is —H and/or -[A]-COOH if ƒ is an ether function; s=1; t=1; u=1 or 2; E is resulting from an amino acid, a diamine, an amino diacid, a triamine, an amino alcohol or an amino diol; -[AA] is resulting from phenylalanine, phenylglycine, tyrosine or tryptophan.

In one embodiment, L is —H and/or -[A]-COOH if ƒ is an ether function; s=1; t=1; u=1; E is resulting from an amino acid, a diamine, an amino diacid, a triamine, an amino alcohol or an amino diol; -[AA] is resulting from phenylalanine, phenylglycine, tyrosine or tryptophan.

In one embodiment, L is —H and/or -[A]-COOH if ƒ is an ether function; s=1; t=1; u=1 or 2; E is resulting from an amino acid, ethylenediamine or ethanolamine; -[AA] is resulting from phenylalanine, phenylglycine, tyrosine or tryptophan.

In one embodiment, L is —H and/or -[A]-COOH if ƒ is an ether function; s=1; t=1; u=1 or 2; E is resulting from an amino acid, a diamine, an amino diacid, a triamine, an amino alcohol or an amino diol; -[AA] is resulting from phenylalanine.

In one embodiment, —R₁ is chosen from the radicals of formula —N(L)ₛ([E]-(o-[AA])ᵤ)ₜ in which L, E, AA, s, t, u and o have the meanings given above, and X is a radical —C═O—.

In one embodiment, the substituted anionic compound corresponds to formula I in which:
3) X is a radical —CO—,
4) R₁ is a radical —N(L)ₛ([E]-(o-[AA])ᵤ)ₜ, L, E, AA, s, t, u and o being as defined above.

In one embodiment, L is —H.

In one embodiment, L is a linear or branched alkyl radical comprising from 1 to 4 carbon atoms.

In one embodiment, s=1.

In one embodiment, t=1.

In one embodiment, E is resulting from an amino acid, an amino diacid, a diamine, a triamine, an amino alcohol or an amino diol.

In one embodiment, E is resulting from an amino acid, ethylenediamine or ethanolamine.

In one embodiment, -[AA] is resulting from phenylalanine, phenylglycine, tyrosine or tryptophan.

In one embodiment, -[AA] is resulting from phenylalanine.

In one embodiment, u=1 or 2.

In one embodiment, L is —H; s=1; t=1; u=1 or 2; E is resulting from an amino acid, a diamine, an amino diacid, a triamine, an amino alcohol or an amino diol; -[AA] is resulting from phenylalanine, phenylglycine, tyrosine or tryptophan.

In one embodiment, L is —H; s=1; t=1; u=1; E is resulting from an amino acid, from a diamine, an amino diacid, a triamine, an amino alcohol or an amino diol; -[AA] is resulting from phenylalanine, phenylglycine, tyrosine or tryptophan.

In one embodiment, L is —H; s=1; t=1; u=1 or 2; E is resulting from an amino acid, ethylenediamine or ethanolamine; -[AA] is resulting from phenylalanine, phenylglycine, tyrosine or tryptophan.

In one embodiment, L is —H; s=1; t=1; u=1 or 2; E is resulting from an amino acid, an amino diacid, a diamine, a triamine, an amino alcohol or an amino diol; -[AA] is resulting from phenylalanine.

According to one embodiment, the substituted anionic compound corresponds to formula I in which Z is a radical —CH₂— and X is a radical —CH₂— and is chosen from the compounds of formula II:

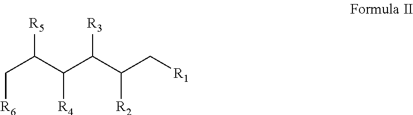

in which:
R₂, R₃, R₄, R₅, R₆, A and p have the values given in the definition of formula I, and
3) ƒ is an ether function;
4) R₁ is a radical —N(L)ₛ[AA], with -[AA] being a radical resulting from an aromatic amino acid or from an aromatic amino acid derivative; s=1 or 2; and
   a. if s=1, L is chosen from the group consisting of —H, —H and/or -[A]-COOH, —H and a linear or branched alkyl radical comprising from 1 to 4 carbon atoms, and
   b. if s=2, L is chosen from —H, —H and/or -[A]-COOH, a linear or branched alkyl radical comprising from 1 to 4 carbon atoms.

According to one embodiment, the substituted anionic compound corresponds to formula I in which Z is a radical —CH₂— and X is a radical —CH₂— and is chosen from the compounds of formula II:

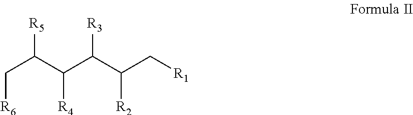

in which:
R₂, R₃, R₄, R₅, R₆, A and p have the values given in the definition of formula I, and
3) ƒ is an ether function, and
4) R₁ is a radical —N(L)ₛ[AA], with -[AA] being a radical resulting from an aromatic amino acid or from an aromatic amino acid derivative; s=1 or 2; and
   a. if s=1, L is chosen from the group consisting of —H, —H and/or -[A]-COOH, and
   b. if s=2, L is chosen from H or —H and/or -[A]-COOH.

According to one embodiment, the substituted anionic compound corresponds to formula I in which Z is a radical —CH₂— and X is a radical —C═O— and is chosen from the compounds of formula III:

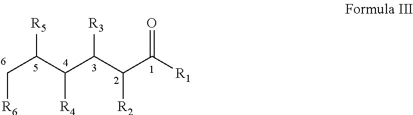

in which
R₂, R₃, R₄, R₅, R₆, A and p have the values given in the definition of formula I, and
3) ƒ is an ether function, and
4) R₁ is a radical —N(L)s[AA], with -[AA] being a radical resulting from an aromatic amino acid or from an aromatic amino acid derivative; s=1; and L is chosen from the group consisting of —H and a linear or branched alkyl radical comprising from 1 to 4 carbon atoms.

According to one embodiment, the substituted anionic compound corresponds to formula I in which Z is a radical —CH₂— and X is a radical —CH₂— and is chosen from the compounds of formula II:

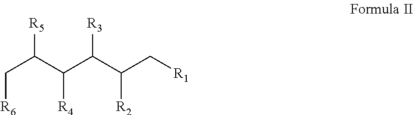

in which:
R₂, R₃, R₄, R₅, R₆, A and p have the values given in the definition of formula I, and
3) ƒ is an ether function, and
4) R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1 or 2; and
   L is chosen from the group consisting of —H, —H and/or -[A]-COOH, —H and a linear or branched alkyl radical comprising from 1 to 4 carbon atoms.

According to one embodiment, the substituted anionic compound corresponds to formula I in which Z is a radical —CH₂— and X is a radical —CH₂— and is chosen from the compounds of formula II:

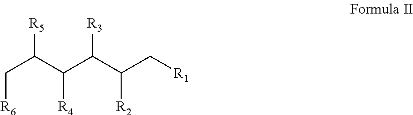

in which:
R₂, R₃, R₄, R₅, R₆, A and p have the values given in the definition of formula I, and
3) ƒ is an ether function, and
4) R₁ is a radical —N(L)ₛ([E]-(o-[AA]), with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1 or 2; and
   L is chosen from the group consisting of —H, —H and/or -[A]-COOH.

According to one embodiment, the substituted anionic compound corresponds to formula I in which Z is a radical —CH₂— and X is a radical —C(O)— and is chosen from the compounds of formula III:

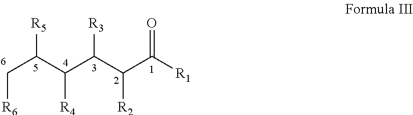

in which
R₂, R₃, R₄, R₅, R₆, A and p have the values given in the definition of formula I, and
3) ƒ is an ether function, and
4) R₁ is a radical —N(L)ₛ([E]-(o-[AA]) with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1; and L is chosen from —H and a linear or branched alkyl radical comprising from 1 to 4 carbon atoms.

According to one embodiment, the substituted anionic compound corresponds to formula I in which Z is a radical —CH₂— and X is a radical —C═O— and is chosen from the compounds of formula III:

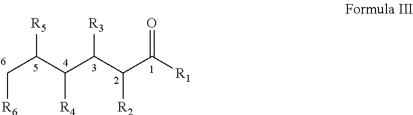

in which
R₂, R₃, R₄, R₅, R₆, A and p have the values given in the definition of formula I, and
3) ƒ is an ether function, and
4) R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1; and L is —H.

Most particularly, in the preceding seven embodiments, the radical -[AA] is resulting from an aromatic amino acid and more particularly from phenylalanine.

According to one embodiment, the substituted anionic compound is chosen from the compounds of formula I in which Z is a radical —CH₂— and R₄ is resulting from a saccharide backbone formed from a discrete number n−1 of glucose saccharide units and is represented by formula IV:

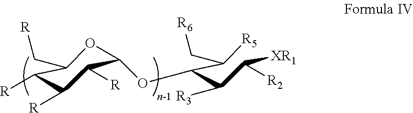

in which
R₁, R₂, R₃, R₅, R₆, X, A and n have the values given in the definition of formula I, and

### R is —OH or -ƒ-[A]-COOH.

According to one embodiment, the substituted anionic compound corresponds to formula IV:

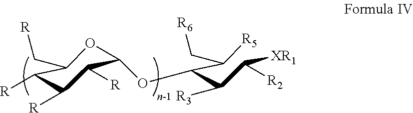

in which
R₂, R₃, R₅, R₆, A and n have the values given in the definition of formula I, and
R is —OH or -ƒ-[A]-COOH, and
X is a radical —CH₂—,
ƒ is an ether function;
R₁ is a radical —N(L)s[AA], with -[AA] being a radical resulting from an aromatic amino acid or an aromatic amino acid derivative; s=1 or 2; and
   c. if s=1, L is chosen from the group consisting of —H, —H and/or -[A]-COOH, —H and a linear or branched alkyl radical comprising from 1 to 4 carbon atoms, and
   d. if s=2, L is chosen from H, —H and/or -[A]-COOH, a linear or branched alkyl radical comprising from 1 to 4 carbon atoms.

According to one embodiment, the substituted anionic compounds correspond to formula IV:

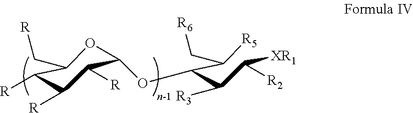

in which
R₂, R₃, R₅, R₆, A and n have the values given in the definition of formula I, and
R is —OH or -ƒ-[A]-COOH, and
X is a radical —CH₂—,
ƒ is an ether function, and
R₁ is a radical —N(L)ₛ[AA], with -[AA] being a radical resulting from an aromatic amino acid or an aromatic amino acid derivative; s=1 or 2; and
   c. if s=1, L is chosen from the group consisting of —H, —H and/or -[A]-COOH, and
   d. if s=2, L is chosen from H or —H and/or -[A]-COOH.

According to one embodiment, the substituted anionic compound corresponds to formula IV:

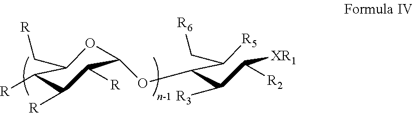

in which
R₂, R₃, R₅, R₆, A and n have the values given in the definition of formula I, and
R is —OH or -ƒ-[A]-COOH, and
X is a radical —C═O—,
ƒ is an ether function, and
R₁ is a radical —N(L)ₛ[AA], with -[AA] being a radical resulting from an aromatic amino acid or from an aromatic amino acid derivative; s=1; and L is chosen from the group consisting of —H and a linear or branched alkyl radical comprising from 1 to 4 carbon atoms.

According to one embodiment, the substituted anionic compound corresponds to formula IV:

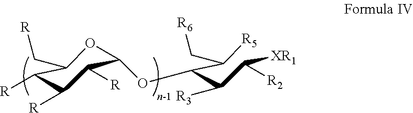

in which
R₂, R₃, R₅ and R₆ have the values given in the definition of formula I, and
R is —OH or -ƒ-[A]-COOH, and
n=3,
-[A]- is a radical —CH₂—,
X is a radical —CH₂—,
ƒ is an ether function, and
R₁ is a radical —N(L)ₛ[AA], with -[AA] being a radical resulting from phenylalanine; s=1 or 2; and L is —H.

According to one embodiment, the substituted anionic compound corresponds to formula IV:

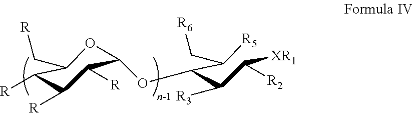

in which
R₂, R₃, R₅, R₆, A and n have the values given in the definition of formula I, and
R is —OH or -ƒ-[A]-COOH, and
X is a radical —CH₂—,
ƒ is an ether function, and
R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1 or 2; and
L is chosen from the group consisting of —H, —H and/or -[A]-COOH, —H and a linear or branched alkyl radical comprising from 1 to 4 carbon atoms.

According to one embodiment, the substituted anionic compound corresponds to formula IV:

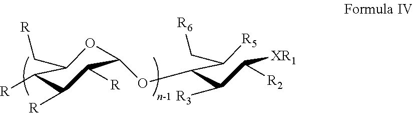

in which
R₂, R₃, R₅, R₆, A and n have the values given in the definition of formula I, and
R is —OH or -ƒ-[A]-COOH, and
X is a radical —CH₂—,
ƒ is an ether function, and
R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1 or 2; and
L is chosen from the group consisting of —H, —H and/or -[A]-COOH.

According to one embodiment, the substituted anionic compound corresponds to formula IV:

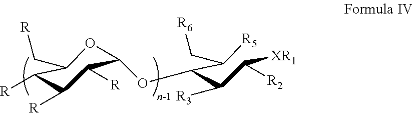

in which
R₂, R₃, R₅, R₆, A and n have the values given in the definition of formula I, and
R is —OH or -ƒ-[A]-COOH, and
X is a radical —C═O—,
ƒ is an ether function, and
R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1; and L is chosen from —H and a linear or branched alkyl radical comprising from 1 to 4 carbon atoms.

According to one embodiment, the substituted anionic compound corresponds to formula IV:

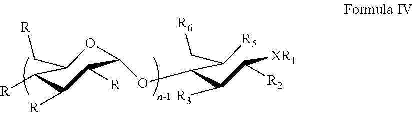

in which
R₂, R₃, R₅, R₆, A and n have the values given in the definition of formula I, and
R is —OH or -ƒ-[A]-COOH, and
X is a radical —C═O—,
ƒ is an ether function, and
R₁ is a radical —N(L)ₛ([E]-(o-[AA])ᵤ)ₜ, with —N((L)ₛ-([E]-(o-)ᵤ) being resulting from an amino acid, an amino diacid, a diamine, a triamine, an amino alcohol or an amino diol; o is an amide, carbamate or carbamide function; -[AA] is a radical resulting from an aromatic amino acid or an aromatic amino acid derivative; u=1 or 2; t=1; s=1; and L is —H.
   Most particularly, in the preceding eight embodiments, the radical -[AA] is resulting from an aromatic amino acid and more particularly from phenylalanine.

The substituted anionic compounds comprise at least one radical -ƒ-[A]-COOH. The radical(s) -ƒ-[A]-COOH may be introduced onto the saccharide units by statistical grafting.

In one embodiment, the substituted anionic compounds are chosen from the substituted anionic compounds in which the radicals -ƒ-[A]-COOH are obtained by grafting at precise positions on the saccharide units via a process involving steps of protection/deprotection of the alcohol or carboxylic acid groups naturally borne by the saccharide units. The strategy leads to selective grafting, especially regioselective grafting, of the substituents onto the saccharide units. The protecting groups include, without limitation, those described in the book (Wuts, P. G. M. et al., Greene's Protective Groups in Organic Synthesis, 2007).

The saccharide precursor of the substituted anionic compound may be obtained by degradation of a high molecular weight polysaccharide. The degradation routes include, without limitation, chemical degradation and/or enzymatic degradation.

The saccharide precursor of the substituted anionic compound may also be obtained by formation of glycoside bonds between monosaccharide or oligosaccharide molecules using a chemical or enzymatic coupling strategy, and the saccharide then obtained comprises a reductive end. The coupling strategies include those described in the publication (Smooth, J. T. et al., Advances in Carbohydrate Chemistry and Biochemistry, 2009, 62, 162-236) and in the book (Lindhorst, T. K., Essentials of Carbohydrate Chemistry and Biochemistry, 2007, 157-209). The coupling reactions may be performed in solution on a solid support. The saccharide molecules before coupling may bear substituents of interest and/or may be functionalized once coupled together statistically or regio selectively.

Thus, by way of example, the substituted anionic compounds may be obtained according to one of the following processes:
the grafting of radicals -ƒ-[A]-COOH by statistical grafting onto the saccharide backbone coupled to the open saccharide unit bearing a radical -[AA]
one or more glycosylation steps between monosaccharide or oligosaccharide molecules bearing radicals -ƒ-[A]-COOH and the saccharide backbone coupled to the open saccharide unit bearing a radical -[AA]
one or more glycosylation steps between one or more monosaccharide or oligosaccharide molecules bearing radicals -ƒ-[A]-COOH and one or more monosaccharide or oligosaccharide molecules and the saccharide backbone coupled to the open saccharide unit bearing a radical -[AA]
one or more steps of introducing protecting groups onto alcohols or acids naturally borne by the saccharide backbone coupled to the open saccharide unit bearing a radical -[AA] followed by one or more grafting reactions to introduce radicals -ƒ-[A]-COOH and finally a step of removing the protecting groups
one or more steps of glycosylation between one or more monosaccharide or oligosaccharide molecules bearing protecting groups on alcohols or acids naturally borne by the saccharide units, one of these saccharides bearing a radical -[AA], one or more grafting steps to introduce radicals -ƒ-[A]-COOH onto the backbone obtained, and then a step of removing the protecting groups,
one or more steps of glycosylation between one or more monosaccharide or oligosaccharide molecules bearing protecting groups on alcohols or acids naturally borne by the saccharide units, and one or more monosaccharide or oligosaccharide molecules, one of these molecules bearing a radical -[AA], one or more grafting steps to introduce -ƒ-[A]-COOH, and then a step of removing the protecting groups,
a step of protecting the reductive chain end present on the precursor of the substituted anionic compound, a step of introducing radicals -ƒ-[A]-COOH onto the saccharide units, a step of deprotecting the reductive chain end and then a step of introducing a radical —N(L)ₛ([E]-(o-[AA])ᵤ)ₜ or —N(L)ₛ[AA] by reaction with the reductive chain end.

The substituted anionic compounds, isolated or as a mixture, may be separated and/or purified in various ways, especially after they have been obtained via the processes described above.

Mention may be made in particular of chromatographic methods, especially “preparative” methods, such as:
flash chromatography, especially on silica, and
chromatographies of the HPLC type (hi-performance liquid chromatography), in particular RP-HPLC (reverse-phase HPLC).

Selective precipitation methods may also be used.

### PART A: SYNTHESIS OF THE SUBSTITUTED ANIONIC COMPOUNDS

### Example A1

### Substituted Anionic Compound A1

Compound 1: product obtained by reductive amination reaction between the reductive chain end of maltotriose and L-phenylalanine methyl ester according to the modified procedure of Sisu, E. et al, Central European Journal of Chemistry 2008, 7 (1), 66-73.

To a solution of L-phenylalanine methyl ester (11.13 g, 62.18 mmol) in DMSO (210 mL) at 50° C. are successively added para-toluenesulfonic acid monohydrate (11.83 g, 62.18 mmol), maltotriose (CarboSynth) (10.45 g, 20.72 mmol), acetic acid (2.1 mL) and sodium cyanoborohydride (13.02 g, 207.18 mmol). After stirring at 50° C. for 4 days, acetone is added to the reaction medium and the precipitate formed is isolated by filtration. The precipitate obtained is dissolved in a minimum volume of water and is reprecipitated by adding acetone. After filtration, the mixture is isolated and then dried under vacuum.

Yield: 11.78 g (85%).

The product is analyzed by ¹H NMR under conditions of hydrolysis of methyl ester.

¹H NMR (D₂O+NaOD+DCI, ppm): 7.25-7.12 (m, 5H); 5.18 (d, 1H, J=3.8 Hz); 4.89 (d, 1H, J=3.8 Hz); 4.24 (dd, 1H, J=6.6 and 6.6 Hz); 3.96 (m, 1H); 3.80-3.72 (m, 3H); 3.67-3.36 (m, 13H); 3.24-3.08 (m, 5H).

Only the product with the hydrolyzed methyl ester is observed on LC/MS analysis.

LC/MS (ESI-ES+): 654.5 (calculated ([M+H]⁺): 654.3).

### Substituted Anionic Compound A1

Compound 1 (11.7 g) is dissolved in 20 mL of water and the solution is heated to 55° C. 3 mL of 10N NaOH are added to this solution and the mixture is stirred at 55° C. for 10 minutes, and the pH of the solution is then adjusted to 7 by adding 2.2 mL of an HCl solution (37%). The reaction mixture is then brought to 65° C. and sodium chloroacetate (36.7 g, 315.1 mmol) is then added. After stirring for 60 minutes, 32 mL of 10 N NaOH are added dropwise. 3 hours after the start of addition of NaOH, sodium chloroacetate (18.4 g, 157.5 mmol) is added. After stirring for 20 minutes, a further 16 mL of 10N NaOH are added dropwise. After stirring for 60 minutes, the reaction medium is diluted with water, neutralized with acetic acid and then purified by ultrafiltration on a 1 kDa PES membrane against NaCl (0.9%) and water.

The concentration of substituted anionic compound A1 in the final solution is determined by means of the dry extract.

According to the dry extract: [substituted anionic compound A1]=11.9 mg/g

The degree of substitution with sodium methylcarboxylate per saccharide unit is determined by integration relative to the signal for the carbonyl of the methyl carboxylate unit with the signals for the carbons of the aromatic ring of phenylalanine by ¹³C NMR. The degree of substitution with sodium methylcarboxylate per saccharide unit is 1.9.

### Example A2

### Substituted Anionic Compound A2

### Substituted Anionic Compound A2

Compound 1 (9 g) is dissolved in 12 mL of water and the solution is heated to 65° C. Sodium chloroacetate (18.2 g, 156 mmol) is added. After total dissolution of the chloroacetate, 16 mL of 10N NaOH are added dropwise. 3 hours after the start of addition of NaOH, the reaction medium is diluted with water, neutralized with acetic acid and then purified by ultrafiltration on a 1 kDa PES membrane against NaCl (0.9%) and water.

The concentration of substituted anionic compound A2 in the final solution is determined by means of the dry extract.

According to the dry extract: [substituted anionic compound A2]=13.1 mg/g The degree of substitution with sodium methylcarboxylate per saccharide unit is determined by integration relative to the signal for the carbonyl of the methyl carboxylate unit with the signals for the carbons of the aromatic ring of phenylalanine by 13C NMR. The degree of substitution with sodium methylcarboxylate per saccharide unit is 1.56.

### PART B: PREPARATION OF THE SOLUTIONS

### B1

### Novolog® Rapid Insulin Analog Solution at 100 IU/mL

This solution is a commercial solution of aspart insulin from Novo Nordisk sold under the name Novolog®. This product is a rapid insulin analog.

### B2

### Humalog® Rapid Insulin Analog Solution at 100 IU/mL

This solution is a commercial solution of lispro insulin from Eli Lilly sold under the name Humalog®. This product is a rapid insulin analog.

### B3

### Humulin®R Human Insulin Solution at 100 IU/mL

This solution is a commercial solution of human insulin from Eli Lilly sold under the name Humulin® R. This product is a human insulin formulation.

### B4

### Apidra® Rapid Insulin Analog Solution at 100 IU/mL

This solution is a commercial solution of glulisine insulin from Sanofi sold under the name Apidra®. This product is a rapid insulin analog.

### B5

### Preparation of a 1.188 M Sodium Citrate Solution

The sodium citrate solution is obtained by dissolving 9.0811 g of sodium citrate (30.9 mmol) in 25 mL of water in a graduated flask. The pH is adjusted to 7.4 by adding 1 mL of 1 M HCl. The solution is filtered through a 0.22 μm membrane.

### B6

### Preparation of a Solution of Lispro Insulin at 100 IU/mL in the Presence of the Substituted Anionic Compound A1

For a final volume of 100 mL of formulation, with a [substituted anionic compound A1]/[lispro insulin] mass ratio of 2.0, the various reagents are added in the amounts specified below and in the following order:

| | | |
|---|---|---|
| Lyophilized compound (substituted anionic compound A1) | 730 | mg |
| Commercial solution of Humalog ® | 100 | mL |

The final pH is adjusted to 7.4±0.4.

The clear solution is filtered through a 0.22 μm membrane and stored at 4° C.

### B7

### Preparation of a Solution of Lispro Insulin at 100 IU/mL in the Presence of the Substituted Anionic Compound A1 and Citrate

For a final volume of 100 mL of formulation, with a [substituted anionic compound A1]/[lispro insulin] mass ratio of 2.0 and a concentration of 9.3 mM of citrate, the various reagents are added in the amounts specified below and in the following order:

| | | |
|---|---|---|
| Lyophilized compound (substituted anionic compound A1) | 730 | mg |
| Commercial solution of Humalog ® | 100 | mL |
| Sodium citrate solution at 1.188M | 783 | μL |

The final pH is adjusted to 7.4±0.4.

The clear solution is filtered through a 0.22 μm membrane and stored at 4° C.

### B8

### Preparation of a Solution of Human Insulin at 100 IU/mL in the Presence of the Substituted Anionic Compound A1 and Citrate

For a final volume of 100 mL of formulation, with a [substituted anionic compound A1]/[human insulin] mass ratio of 2.0 and a concentration of 9.3 mM of citrate, the various reagents are added in the amounts specified below and in the following order:

| | | |
|---|---|---|
| Lyophilized compound (substituted anionic compound A1) | 730 | mg |
| Commercial solution of Humulin ® R | 100 | mL |
| Sodium citrate solution at 1.188M | 783 | μL |

The final pH is adjusted to 7.4±0.4.

The clear solution is filtered through a 0.22 μm membrane and stored at 4° C.

### B9

### Preparation of a Solution of Aspart Insulin at 100 IU/mL in the Presence of the Substituted Anionic Compound A1 and Citrate

For a final volume of 100 mL of formulation, with a [substituted anionic compound A1]/[aspart insulin] mass ratio of 2.0 and a concentration of 9.3 mM of citrate, the various reagents are added in the amounts specified below and in the following order:

| | | |
|---|---|---|
| Lyophilized compound (substituted anionic compound A1) | 730 | mg |
| Commercial solution of Novolog ® | 100 | mL |
| Sodium citrate solution at 1.188M | 783 | μL |

The final pH is adjusted to 7.4±0.4.

The clear solution is filtered through a 0.22 μm membrane and stored at 4° C.

### B10

### Preparation of a Solution of Glulisine Insulin at 100 IU/mL in the Presence of the Substituted Anionic Compound A1 and Citrate

For a final volume of 100 mL of formulation, with a [substituted anionic compound A1]/[glulisine insulin] mass ratio of 2.0 and a concentration of 9.3 mM of citrate, the various reagents are added in the amounts specified below and in the following order:

| | | |
|---|---|---|
| Lyophilized compound (substituted anionic compound A1) | 730 | mg |
| Commercial solution of Apidra ® | 100 | mL |
| Sodium citrate solution at 1.188M | 783 | μL |

The final pH is adjusted to 7.4±0.4.

The clear solution is filtered through a 0.22 μm membrane and stored at 4° C.

### Example B11

### Preparation of a Solution of Substituted Anionic Compound A1 at 315 mg/mL

The substituted anionic compound A1 obtained in example A1 is lyophilized. An amount of lyophilizate is dissolved in water for injection so as to obtain a solution at 360 mg/mL at pH 7.5 of substituted anionic compound A1.

### Example B12

### Preparation of a Solution of Lispro Insulin at 100 IU/mL in the Presence of the Substituted Anionic Compound A2

For a final volume of 100 mL of formulation, with a [substituted anionic compound A2]/[lispro insulin] mass ratio of 2.0, the various reagents are added in the amounts specified below and in the following order:

| | | |
|---|---|---|
| Lyophilized compound (substituted anionic compound A2) | 730 | mg |
| Commercial solution of Humalog ® | 100 | mL |

The final pH is adjusted to 7.4±0.4.

The clear solution is filtered through a 0.22 μm membrane and stored at 4° C.

### B13

### Preparation of a Solution of Lispro Insulin at 100 IU/mL in the Presence of the Substituted Anionic Compound A2 and Citrate

For a final volume of 100 mL of formulation, with a [substituted anionic compound A2]/[lispro insulin] mass ratio of 2.0 and a concentration of 9.3 mM of citrate, the various reagents are added in the amounts specified below and in the following order:

| | | |
|---|---|---|
| Lyophilized compound (substituted anionic compound A2) | 730 | mg |
| Commercial solution of Humalog ® | 100 | mL |
| Sodium citrate solution at 1.188M | 783 | μL |

The final pH is adjusted to 7.4±0.4.

The clear solution is filtered through a 0.22 μm membrane and stored at 4° C.

### B14

### Preparation of a Solution of Human Insulin at 100 IU/mL in the Presence of the Substituted Anionic Compound A2 and Citrate

For a final volume of 100 mL of formulation, with a [substituted anionic compound A2]/[human insulin] mass ratio of 2.0 and a concentration of 9.3 mM of citrate, the various reagents are added in the amounts specified below and in the following order:

| | | |
|---|---|---|
| Lyophilized compound (substituted anionic compound A2) | 730 | mg |
| Commercial solution of Humulin ® R | 100 | ml |
| Sodium citrate solution at 1.188M | 783 | μL |

The final pH is adjusted to 7.4±0.4.

The clear solution is filtered through a 0.22 μm membrane and stored at 4° C.

### B15

### Preparation of a Solution of Aspart Insulin at 100 IU/mL in the Presence of the Substituted Anionic Compound A2 and Citrate

For a final volume of 100 mL of formulation, with a [substituted anionic compound A2]/[aspart insulin] mass ratio of 2.0 and a concentration of 9.3 mM of citrate, the various reagents are added in the amounts specified below and in the following order:

| | | |
|---|---|---|
| Lyophilized compound (substituted anionic compound A2) | 730 | mg |
| Commercial solution of Novolog ® | 100 | ml |
| Sodium citrate solution at 1.188M | 783 | μL |

The final pH is adjusted to 7.4±0.4.

The clear solution is filtered through a 0.22 μm membrane and stored at 4° C.

### B16

### Preparation of a Solution of Glulisine Insulin at 100 IU/mL in the Presence of the Substituted Anionic Compound A2 and Citrate

For a final volume of 100 mL of formulation, with a [substituted anionic compound A2]/[glulisine insulin] mass ratio of 2.0 and a concentration of 9.3 mM of citrate, the various reagents are added in the amounts specified below and in the following order:

| | | |
|---|---|---|
| Lyophilized compound (substituted anionic compound A2) | 730 | mg |
| Commercial solution of Apidra ® | 100 | mL |
| Sodium citrate solution at 1.188M | 783 | μL |

The final pH is adjusted to 7.4±0.4.

The clear solution is filtered through a 0.22 μm membrane and stored at 4° C.

### PART C: TEST OF DISSOLUTION OF HUMAN INSULIN AT ITS ISOELECTRIC POINT

Human insulin has an isoelectric point of 5.3. At a pH of 5.3, human insulin precipitates at a concentration of greater than or equal to 10 IU/mL (0.36 mg/mL) A test demonstrating an interaction between human insulin and the substituted anionic compounds at the isoelectric point was performed. If an interaction exists between human insulin and the substituted anionic compound, it is then possible to dissolve the human insulin at its isoelectric point.

A solution of human insulin at 500 IU/mL is prepared. A mixture between a solution of human insulin and the solution of substituted anionic compound A1 prepared in example B11 is prepared to give a solution containing 100 IU/mL of human insulin and the desired concentration of anionic compound A1. The pH is adjusted to 5.3 by adding hydrochloric acid or sodium hydroxide as a function of the pH reach following mixing between the substituted anionic compound A1 and the human insulin solution.

The appearance of the solution is documented. If the solution is turbid, the substituted anionic compound A1 at the test concentration does not allow dissolution of the human insulin at its isoelectric point. If the solution is clear, the substituted anionic compound A1 allows dissolution of the human insulin at the test concentration. In this way, an estimation of the concentration of substituted anionic compound A1 required to dissolve human insulin at its isoelectric point may be determined. The lower this concentration, the greater the affinity of the substituted anionic compound A1 for human insulin. The results obtained are presented in Table 1.

TABLE 1

| Solution | Visual appearance |
|---|---|
| Human insulin alone | Turbid |
| (100 IU/mL) | |
| Human insulin (100 IU/mL) and | Turbid |
| Substituted anionic compound A1 | |
| (10 mg/mL) | |
| Human insulin (100 IU/mL) and | Clear |
| Substituted anionic compound A1 | |
| (50 mg/mL) | |

The results show that the substituted anionic compound A1 allows the dissolution of human insulin at its isoelectric point at a concentration of 50 mg/mL, which is the sign of formation of a complex between the human insulin at its isoelectric point and the substituted anionic compound A1.

### D Pharmacodynamics and Pharmacokinetics

### D1

### Protocol for Measuring the Pharmacodynamics and Pharmacokinetics of the Insulin Solutions

12 domestic pigs weighing about 50 kg, precatheterized in the jugular vein, are fasted for 2.5 hours before the start of the experiment. Within the hour prior to the injection of insulin, three blood samples are collected so as to determine the basal level of glucose and insulin.

The injection of insulin at a dose of 0.09 IU/kg for lispro insulin is performed subcutaneously in the neck, under the animal's ear, using a Novopen insulin pen equipped with a 31 G needle.

Blood samples are then collected every 4 minutes for 20 minutes and then every 10 minutes up to 3 hours. After each sample collection, the catheter is rinsed with a dilute heparin solution.

A drop of blood is collected to determine the glycemia using a glucometer.

The pharmacodynamic curves for glucose expressed as the delta of the basal level of glucose (Delta Glucose) are then plotted and the time required to reach the minimum level of glucose in the blood for each pig is determined and reported as Tmin glucose. The mean of the Tmin glucose values is then calculated.

The remaining blood is collected in a dry tube and is centrifuged to isolate the serum. The insulin levels in the serum samples are measured via the sandwich ELISA immunoenzymatic method for each pig.

The pharmacokinetic curves expressed as the delta of the basal level (Delta Glucose) are then plotted. The time required to reach the maximum insulin concentration in the serum for each pig is determined and reported as Tmax insulin. The mean of the Tmax insulin values is then calculated.

### D2

### Pharmacodynamic and Pharmacokinetic Results for the Insulin Solutions of Examples B2 and B7

| | | Substituted | | |
|---|---|---|---|---|
| | | anionic | Polyanionic | Number of |
| Example | Insulin | compound | compound | pigs |
| B2 | lispro | — | — | 11 |
| B7 | lispro | A1 | Citrate 9.3 mM | 9 |

### BRIEF DESCRIPTION OF THE DRAWINGS

FIG. 1: Delta Glucose (mmol/L) as a function of time after injection (min.). Curve plotted with the squares corresponding to example B7, glucose Tmin=31±6 min, curve plotted with the triangles corresponding to example B2, glucose Tmin=43±8 min.

FIG. 2: Delta Insulin (pmol/L) as a function of time after injection (min.). Curve plotted with the squares corresponding to example B7, insulin Tmax=8±4 min, curve plotted with the triangles corresponding to example B2, insulin Tmax=23±9 min.

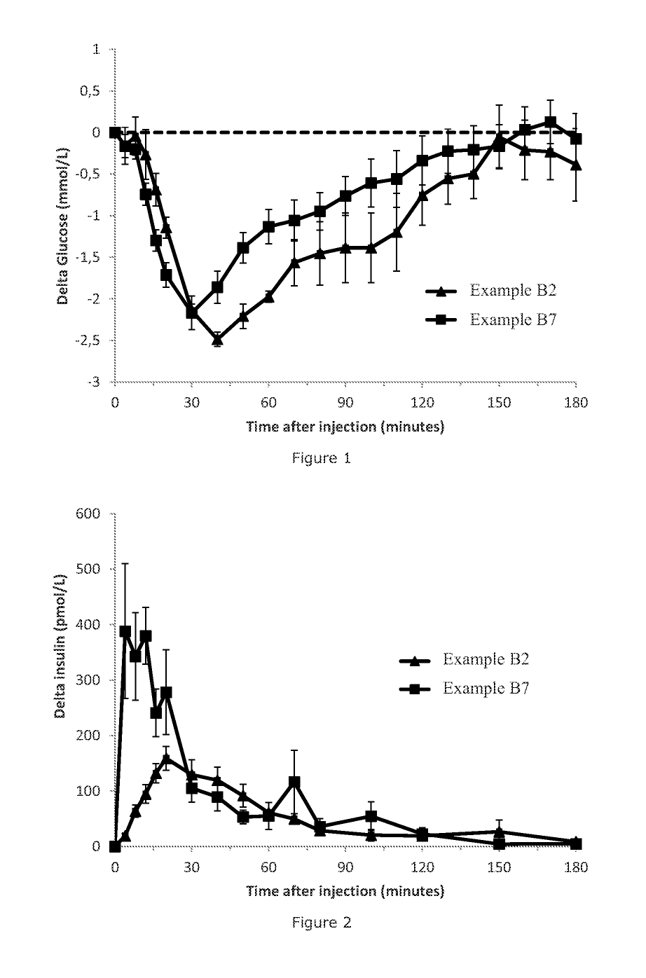

The pharmacodynamic results obtained with the formulations described in examples B2 and B7 are presented in FIG. 1. According to the invention, analysis of these curves shows that the formulation of example B7 comprising the substituted anionic compound A1 and citrate at 9.3 mM as excipients (curve plotted with the squares corresponding to Example B7, Tmin glucose=31±6 min) makes it possible to obtain faster action than that of the Humalog® commercial formulation (lispro insulin) of example B2 (curve plotted with the triangles corresponding to example B2, Tmin glucose=43±8 min).

The pharmacokinetic results obtained with the formulations described in examples B2 and B7 are presented in FIG. 2. According to the invention, analysis of these curves shows that the formulation of example B7 comprising the substituted anionic compound A1 and citrate at 9.3 mM as excipients (curve plotted with the squares corresponding to Example B7, Tmax insulin=8±4 min) induces faster absorption of the lispro insulin than that of the Humalog® commercial formulation (lispro insulin) of example B2 (curve plotted with the triangles corresponding to example B2, Tmax insulin=23±9 min).

1. A composition, in aqueous solution, comprising insulin in hexameric form, at least one substituted anionic compound and at least one polyanionic compound,
said substituted anionic compound consisting of a discrete number n of between 1 and 8 of identical or different saccharide units, linked via identical or different glycoside bonds, said saccharide unit or one of said saccharide units being in open, oxidized or reduced form, said compound comprising salifiable carboxyl groups and said substituted anionic compound bearing on its reductive chain end at least one radical AA resulting from an aromatic amino acid comprising a phenyl or an indole, which may or may not be substituted, or an aromatic amino acid derivative comprising a phenyl or an indole, which may or may not be substituted,
wherein the substituted anionic compound is chosen from the compounds of formula I, said formula I representing the saccharide unit in open form in which at most one from among R₂, R₃, R₄ and R₆ represents a saccharide backbone formed from a discrete number of closed saccharide units: 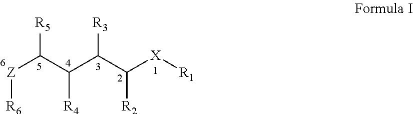
in which
1) Z is either a radical —C═O—, or a radical —CH₂—,
2) X is either a radical —C═O—, or a radical —CH₂—,
3) R₅ is either an —OH radical, or a radical -f-[A]-COOH,
4) R₂, R₃, R₄, R₆, which may be identical or different, are chosen from the group consisting of the radicals —OH, -f-[A]-COOH and at most one from among R₂, R₃, R₄, R₆ is a radical resulting from a saccharide backbone formed from a discrete number n−1 of between 1 and 7 (1≦n−1≦7) of identical or different closed saccharide units, the hydroxyl functions of which may or may not be substituted with a radical -f-[A]-COOH,
5) -[A]- is an at least divalent radical comprising from 1 to 4 carbon atoms chosen from the group consisting of alkyl radicals —(CH₂)x- 1≦x≦4, radicals comprising at least one heteroatom chosen from O, N and S and radicals bearing carboxyl functions and/or -f-[A]-COOH is resulting from an amino acid or an acid alcohol, comprising from 2 to 5 carbon atoms, and is linked to the saccharide units of the compound via a function f;
6) f is chosen from the group consisting of ether, carbamate and amide functions;
7) R₁ is a radical —N(L)ₛ([E]-(o-[AA])ᵤ)ₜ or —N(L)ₛ[AA]
the linker arm -E- is an at least divalent radical comprising from 1 to 10 carbon atoms optionally comprising at least one heteroatom chosen from O, N and S, and optionally bearing carboxyl functions, and/or —N(L)ₛ-([E]-(ₒ-)u) is resulting from an amino acid, a diamine, an amino alcohol, an amino diacid, a triamine, a tetramine, an amino diol or an amino triol comprising from 2 to 12 carbon atoms, the amine functions of which are primary and/or secondary;
-[AA] is resulting from an aromatic amino acid comprising a phenyl or an indole, which may or may not be substituted, or an aromatic amino acid derivative containing a phenyl or an indole, which may or may not be substituted,
o is an amide, carbamate or carbamide function,
u=1, 2 or 3 and
when R₁ is a radical —N(L)ₛ([E]-(o-[AA])ᵤ)ₜ and
when X is a radical —C═O— then
s=0 or 1, t=1 or 2 and s+t=2;
L is chosen from the group consisting of
—H, and
a linear or branched alkyl radical comprising from 1 to 4 carbon atoms, and
when X is a radical —CH₂—, then
s=0, 1 or 2, t=1 or 2 and s+t=2 or 3;
if s=1, L is chosen from the group consisting of
—H, and
—H and/or -[A]-COOH if f as defined in point 6) is an ether function,
—H and/or —CO—NH-[A]-COOH if f as defined in point 6 is a carbamate function, and
a linear or branched alkyl radical comprising from 1 to 4 carbon atoms, and
if s=2, L is chosen from the group consisting of:
—H, and
—H and/or -[A]-COOH if f as defined in point 6) is an ether function, and
a linear or branched alkyl radical comprising from 1 to 4 carbon atoms, and
when R₁ is a radical —N(L)ₛ[AA] and
when X is a radical —C═O— then s=1 and L is chosen from the group consisting of:
—H,
a linear or branched alkyl radical comprising from 1 to 4 carbon atoms,
when X is a radical —CH₂—, then s=1 or 2 and
if s=1, L is chosen from the group consisting of:
—H, and
—H and/or -[A]-COOH if f as defined in point 6) is an ether function,
—H and/or —CO—NH-[A]-COOH if f as defined in point 6) is a carbamate function, and
a linear or branched alkyl radical comprising from 1 to 4 carbon atoms, and
if s=2, L is chosen from the group consisting of
—H, and
—H and/or -[A]-COOH if f as defined in point 6) is an ether function, and
a linear or branched alkyl radical comprising from 1 to 4 carbon atoms, and
8) the degree of substitution represented by p is the number of carboxylate functions per saccharide unit, said carboxylate functions optionally being carboxylate functions that are naturally present on the saccharide units, being resulting from substitution with radicals -[A]-COOH and/or radicals -[AA] and 6≧p≧0.1,
and the acid functions being in the form of salts of alkali metal cations chosen from the group consisting of Na⁺ and K⁺.

2. The composition as claimed in claim 1, wherein the substituted anionic compound is chosen from the compounds of formula I in which the radical -[AA] is resulting from an aromatic amino acid comprising a phenyl or an indole, which may or may not be substituted, or from an aromatic amino acid derivative comprising a phenyl or an indole, which may or may not be substituted.

3. The composition as claimed in claim 1, wherein the substituted anionic compound is chosen from the compounds of formula I in which the radical -[AA] is resulting from an aromatic amino acid comprising a phenyl or an indole, which may or may not be substituted, chosen from the group consisting of phenylalanine, alpha-methylphenylalanine, 3,4-dihydroxyphenylalanine, alpha-phenylglycine, 4-hydroxyphenylglycine, 3,5-phenylglycine, tyrosine, alpha-methyltyrosine, O-methyltyrosine and tryptophan.

4. The composition as claimed in claim 1, wherein the substituted anionic compound is chosen from the compounds of formula I in which the radical -f-[A]-COOH is chosen from the group consisting of the following sequences, f having the meaning given above: 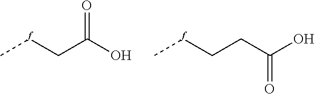
or salts thereof of alkali metal cations chosen from the group consisting of Na⁺ and K⁺.

5. The composition as claimed in claim 1, wherein the substituted anionic compound is chosen from the compounds of formula I in which 3.5≧p≧0.1.

6. The composition as claimed in claim 1, wherein the substituted anionic compound is chosen from the compounds of formula I in which at most one from among R₂, R₃, R₄ and R₆ is a radical resulting from a backbone formed from a discrete number n−1 of identical or different saccharide units and said saccharide units are chosen from the group consisting of pentoses, hexoses, uronic acids and N-acetylhexosamines.

7. The composition as claimed in claim 1, wherein the substituted anionic compound is chosen from the compounds of formula I in which the saccharide unit in open form is resulting from a saccharide unit chosen from the group consisting of pentoses, hexoses, uronic acids and N-acetylhexosamines.

8. The composition as claimed in claim 1, wherein the substituted anionic compound is chosen from the compounds of formula I in which the radical R₁ is chosen from the radicals of formula —N(L)ₛ-([E]-(o-[AA])ₜ.

9. The composition as claimed wherein the radical —N(L)ₛ-([E]-(o-[AA])ₜ is chosen from radicals in which —N(L)ₛ-([E]-(o-)ᵤ)- is an at least divalent radical resulting from an amino acid comprising from 2 to 12 carbon atoms.

10. The composition as claimed in wherein the substituted anionic compound is chosen from the compounds of formula I in which the radical R₁ is chosen from the radicals of formula —N(L)ₛ[AA].

11. The composition as claimed in claim 1, wherein the substituted anionic compound is chosen from the compounds of formula IV: 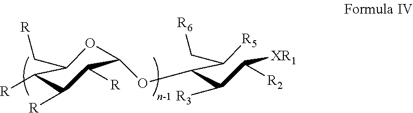
in which
R is —OH or -f-[A]-COOH.

12. The composition as claimed in claim 1, wherein the mole ratios of substituted anionic compound/insulin are between 0.6 and 75.

13. The composition as claimed in claim 1, wherein the insulin is human insulin.

14. The composition as claimed in claim 1, wherein the insulin is an insulin analog.

15. The composition as claimed in claim 1, wherein the polyanionic compound has affinity for zinc lower than the affinity of insulin for zinc and affinity for calcium defined by a dissociation constant Kd_{Ca}=[PNP compound]^{r}[Ca²⁺]^{s}/[(PNP compound)ᵣ-(Ca²⁺)ₛ] is less than or equal to 10^{-1.5}.

16. The composition as claimed in claim 1, wherein the polyanionic compound is an anionic molecule chosen from the group consisting of citric acid, aspartic acid, glutamic acid, malic acid, tartaric acid, succinic acid, adipic acid, oxalic acid, phosphate, polyphosphoric acids, such as triphosphate, and the Na⁺, K⁺, Ca²⁺ or Mg²⁺ salts thereof.

17. The composition as claimed in claim 1, wherein the polyanionic compound is chosen from anionic compounds consisting of a saccharide backbone formed from a discrete number u between 1 and 8 (1≦u≦8) of saccharide units, said saccharide units being chosen from the group consisting of hexoses, in cyclic form or in reduced open form, which may be identical or different, linked via identical or different glycoside bonds substituted with carboxyl groups, and salts thereof.

18. A method for preparing a human insulin formulation with an insulin concentration of between 240 and 3000 μM (40 and 500 IU/mL), whose delay of action in humans is less than that of the reference formulation at the same insulin concentration in the absence of substituted anionic compound and of polyanionic compound, wherein it comprises: (1) a step of adding to said formulation at least one substituted anionic compound,
wherein the substituted anionic compound is chosen from the compounds of formula I, said formula I representing the saccharide unit in open form in which at most one from among R₂, R₃, R₄ and R₆ represents a saccharide backbone formed from a discrete number of closed saccharide units: 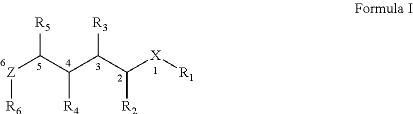
in which
1) Z is either a radical —C═O—, or a radical —CH₂—,
2) X is either a radical —C═O—, or a radical —CH₂—,
3) R₅ is either an —OH radical, or a radical -f-[A]-COOH,
4) R₂, R₃, R₄, R₆, which may be identical or different, are chosen from the group consisting of the radicals —OH, -f-[A]-COOH and at most one from among R₂, R₃, R₄, R₆ is a radical resulting from a saccharide backbone formed from a discrete number n−1 of between 1 and 7 (1≦n−1≦7) of identical or different closed saccharide units, the hydroxyl functions of which may or may not be substituted with a radical -f-[A]-COOH,
5) -[A]- is an at least divalent radical comprising from 1 to 4 carbon atoms chosen from the group consisting of alkyl radicals —(CH₂)x- 1≦x≦4, radicals comprising at least one heteroatom chosen from O, N and S and radicals bearing carboxyl functions and/or -f-[A]-COOH is resulting from an amino acid or an acid alcohol, comprising from 2 to 5 carbon atoms, and is linked to the saccharide units of the compound via a function f;
6) ƒ is chosen from the group consisting of ether, carbamate and amide functions;
7) R₁ is a radical —N(L)ₛ([E]-(o-[AA])ᵤ)ₜ or —N(L)ₛ[AA]
the linker arm -E- is an at least divalent radical comprising from 1 to 10 carbon atoms optionally comprising at least one heteroatom chosen from O, N and S, and optionally bearing carboxyl functions, and/or —N(L)ₛ-([E]-(ₒ-)u) is resulting from an amino acid, a diamine, an amino alcohol, an amino diacid, a triamine, a tetramine, an amino diol or an amino triol comprising from 2 to 12 carbon atoms, the amine functions of which are primary and/or secondary;
-[AA] is resulting from an aromatic amino acid comprising a phenyl or an indole, which may or may not be substituted, or an aromatic amino acid derivative containing a phenyl or an indole, which may or may not be substituted,
o is an amide, carbamate or carbamide function,
u=1, 2 or 3 and
when R₁ is a radical —N(L)ₛ([E]-o-[AA])ᵤ)ₜ and
when X is a radical —C═O— then
s=0 or 1, t=1 or 2 and s+t=2;
L is chosen from the group consisting of
—H, and
a linear or branched alkyl radical comprising from 1 to 4 carbon atoms, and
when X is a radical —CH₂—, then
s=0, 1 or 2, t=1 or 2 and s+t=2 or 3;
if s=1, L is chosen from the group consisting of
—H, and
—H and/or -[A]-COOH if f as defined in point 6) is an ether function,
—H and/or —CO—NH-[A]-COOH if f as defined in point 6 is a carbamate function, and
a linear or branched alkyl radical comprising from 1 to 4 carbon atoms, and
if s=2, L is chosen from the group consisting of:
—H, and
—H and/or -[A]-COOH if f as defined in point 6) is an ether function, and
a linear or branched alkyl radical comprising from 1 to 4 carbon atoms, and
when R₁ is a radical —N(L)ₛ[AA] and
when X is a radical —C═O— then s=1 and L is chosen from the group consisting of:
—H,
a linear or branched alkyl radical comprising from 1 to 4 carbon atoms,
when X is a radical —CH₂—, then s=1 or 2 and
if s=1, L is chosen from the group consisting of:
—H, and
—H and/or -[A]-COOH if f as defined in point 6) is an ether function,
—H and/or —CO—NH-[A]-COOH if f as defined in point 6) is a carbamate function, and
a linear or branched alkyl radical comprising from 1 to 4 carbon atoms, and
if s=2, L is chosen from the group consisting of
—H, and
—H and/or -[A]-COOH if f as defined in point 6) is an ether function, and
a linear or branched alkyl radical comprising from 1 to 4 carbon atoms, and
the degree of substitution represented by p is the number of carboxylate functions per saccharide unit, said carboxylate functions optionally being carboxylate functions that are naturally present on the saccharide units, being resulting from substitution with radicals -[A]-COOH and/or radicals -[AA] and 6≧p≧0.1,
and the acid functions being in the form of salts of alkali metal cations chosen from the group consisting of Na⁺ and K⁺.
and (2) a step of adding to said formulation at least one polyanionic compound.

19. A method for preparing an insulin analog formulation with an insulin concentration of between 240 and 3000 μM (40 and 500 IU/mL), whose delay of action in humans is less than that of the reference formulation at the same insulin concentration in the absence of substituted anionic compound and of polyanionic compound, wherein it comprises: (1) a step of adding to said formulation at least one substituted anionic compound,
wherein the substituted anionic compound is chosen from the compounds of formula I, said formula I representing the saccharide unit in open form in which at most one from among R₂, R₃, R₄ and R₆ represents a saccharide backbone formed from a discrete number of closed saccharide units: 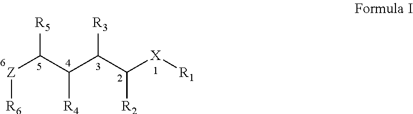
in which
1) Z is either a radical —C═O—, or a radical —CH₂—,
2) X is either a radical —C═O—, or a radical —CH₂—,
3) R₅ is either an —OH radical, or a radical -f-[A]-COOH,
4) R₂, R₃, R₄, R₆, which may be identical or different, are chosen from the group consisting of the radicals —OH, -f-[A]-COOH and at most one from among R₂, R₃, R₄, R₆ is a radical resulting from a saccharide backbone formed from a discrete number n−1 of between 1 and 7 (1≦n−1≦7) of identical or different closed saccharide units, the hydroxyl functions of which may or may not be substituted with a radical -f-[A]-COOH,
5) -[A]- is an at least divalent radical comprising from 1 to 4 carbon atoms chosen from the group consisting of alkyl radicals —(CH₂)x- 1≦x≦4, radicals comprising at least one heteroatom chosen from O, N and S and radicals bearing carboxyl functions and/or -f-[A]-COOH is resulting from an amino acid or an acid alcohol, comprising from 2 to 5 carbon atoms, and is linked to the saccharide units of the compound via a function ƒ;
6) ƒ is chosen from the group consisting of ether, carbamate and amide functions;
7) R₁ is a radical —N(L)ₛ([E]-(o-[AA])ᵤ)ₜ or —N(L)ₛ[AA]
the linker arm -E- is an at least divalent radical comprising from 1 to 10 carbon atoms optionally comprising at least one heteroatom chosen from O, N and S, and optionally bearing carboxyl functions, and/or —N(L)ₛ-([E]-(ₒ-)u) is resulting from an amino acid, a diamine, an amino alcohol, an amino diacid, a triamine, a tetramine, an amino diol or an amino triol comprising from 2 to 12 carbon atoms, the amine functions of which are primary and/or secondary;
-[AA] is resulting from an aromatic amino acid comprising a phenyl or an indole, which may or may not be substituted, or an aromatic amino acid derivative containing a phenyl or an indole, which may or may not be substituted,
o is an amide, carbamate or carbamide function,
u=1, 2 or 3 and
when R₁ is a radical —N(L)ₛ([E]-(o-[AA])ᵤ)ₜ and
when X is a radical —C═O— then
s=0 or 1, t=1 or 2 and s+t=2;
L is chosen from the group consisting of
—H, and
a linear or branched alkyl radical comprising from 1 to 4 carbon atoms, and
when X is a radical —CH₂—, then
s=0, 1 or 2, t=1 or 2 and s+t=2 or 3;
if s=1, L is chosen from the group consisting of
—H, and
—H and/or -[A]-COOH if ƒ as defined in point 6) is an ether function,
—H and/or —CO—NH-[A]-COOH if ƒ as defined in point 6 is a carbamate function, and
a linear or branched alkyl radical comprising from 1 to 4 carbon atoms, and
if s=2, L is chosen from the group consisting of:
—H, and
—H and/or -[A]-COOH if ƒ as defined in point 6) is an ether function, and
a linear or branched alkyl radical comprising from 1 to 4 carbon atoms, and
when R₁ is a radical —N(L)s[AA] and
when X is a radical —C═O— then s=1 and L is chosen from the group consisting of:
—H,
a linear or branched alkyl radical comprising from 1 to 4 carbon atoms,
when X is a radical —CH₂—, then s=1 or 2 and
if s=1, L is chosen from the group consisting of:
—H, and
—H and/or -[A]-COOH if ƒ as defined in point 6) is an ether function,
—H and/or —CO—NH-[A]-COOH if ƒ as defined in point 6) is a carbamate function, and
a linear or branched alkyl radical comprising from 1 to 4 carbon atoms, and
if s=2, L is chosen from the group consisting of
—H, and
—H and/or -[A]-COOH if ƒ as defined in point 6) is an ether function, and
a linear or branched alkyl radical comprising from 1 to 4 carbon atoms, and
the degree of substitution represented by p is the number of carboxylate functions per saccharide unit, said carboxylate functions optionally being carboxylate functions that are naturally present on the saccharide units, being resulting from substitution with radicals -[A]-COOH and/or radicals -[AA] and 6≧p≧0.1,
and the acid functions being in the form of salts of alkali metal cations chosen from the group consisting of Na⁺ and K⁺.
and (2) a step of adding to said formulation at least one polyanionic compound.

20. A pharmaceutical composition comprising a composition as claimed in claim 1, wherein the insulin concentration is between 240 and 3000 μM (40 to 500 IU/mL).

21. A substituted anionic compound chosen from the compounds of formula I, said formula I representing the saccharide unit in open form in which at most one from among R₂, R₃, R₄ and R₆ represents a saccharide backbone formed from a discrete number of closed saccharide units: 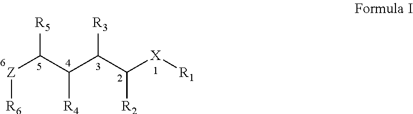
in which
1) Z is either a radical —C═O—, or a radical —CH₂—,
2) X is either a radical —C═O—, or a radical —CH₂—,
3) R₅ is either an —OH radical, or a radical -ƒ-[A]-COOH,
4) R₂, R₃, R₄, R₆, which may be identical or different, are chosen from the group consisting of the radicals —OH, -f-[A]-COOH and at most one from among R₂, R₃, R₄, R₆ is a radical resulting from a saccharide backbone formed from a discrete number n−1 of between 1 and 7 (1≦n−1≦7) of identical or different closed saccharide units, the hydroxyl functions of which may or may not be substituted with a radical -ƒ-[A]-COOH,
5) -[A]- is an at least divalent radical comprising from 1 to 4 carbon atoms chosen from the group consisting of alkyl radicals —(CH₂)x- 1≦x≦4, radicals comprising at least one heteroatom chosen from O, N and S and radicals bearing carboxyl functions and/or -ƒ-[A]-COOH is resulting from an amino acid or an acid alcohol, comprising from 2 to 5 carbon atoms, and is linked to the saccharide units of the compound via a function f;
6) ƒ is chosen from the group consisting of ether, carbamate and amide functions;
7) R₁ is a radical —N(L)ₛ([E]-(o-[AA])ᵤ)ₜ or —N(L)ₛ[AA]
the linker arm -E- is an at least divalent radical comprising from 1 to 10 carbon atoms optionally comprising at least one heteroatom chosen from O, N and S, and optionally bearing carboxyl functions, and/or —N(L)ₛ-([E]-(ₒ-)u) is resulting from an amino acid, a diamine, an amino alcohol, an amino diacid, a triamine, a tetramine, an amino diol or an amino triol comprising from 2 to 12 carbon atoms, the amine functions of which are primary and/or secondary;
-[AA] is resulting from an aromatic amino acid comprising a phenyl or an indole, which may or may not be substituted, or an aromatic amino acid derivative containing a phenyl or an indole, which may or may not be substituted,
o is an amide, carbamate or carbamide function,
u=1, 2 or 3 and
when R₁ is a radical —N(L)ₛ([E]-(o-[AA])ᵤ)ₜ and
when X is a radical —C═O— then
s=0 or 1, t=1 or 2 and s+t=2;
L is chosen from the group consisting of
—H, and
a linear or branched alkyl radical comprising from 1 to 4 carbon atoms, and
when X is a radical —CH₂—, then
s=0, 1 or 2, t=1 or 2 and s+t=2 or 3;
if s=1, L is chosen from the group consisting of
—H, and
—H and/or -[A]-COOH if ƒ as defined in point 6) is an ether function,
—H and/or —CO—NH-[A]-COOH if ƒ as defined in point 6 is a carbamate function, and
a linear or branched alkyl radical comprising from 1 to 4 carbon atoms, and
if s=2, L is chosen from the group consisting of:
—H, and
—H and/or -[A]-COOH if f as defined in point 6) is an ether function, and
a linear or branched alkyl radical comprising from 1 to 4 carbon atoms, and
when R₁ is a radical —N(L)s[AA] and
when X is a radical —C═O— then s=1 and L is chosen from the group consisting of:
—H,
a linear or branched alkyl radical comprising from 1 to 4 carbon atoms,
when X is a radical —CH₂—, then s=1 or 2 and
if s=1, L is chosen from the group consisting of:
—H, and
—H and/or -[A]-COOH if ƒ as defined in point 6) is an ether function,
—H and/or —CO—NH-[A]-COOH if ƒ as defined in point 6) is a carbamate function, and
a linear or branched alkyl radical comprising from 1 to 4 carbon atoms, and
if s=2, L is chosen from the group consisting of
—H, and
—H and/or -[A]-COOH if ƒ as defined in point 6) is an ether function, and
a linear or branched alkyl radical comprising from 1 to 4 carbon atoms, and
8) the degree of substitution represented by p is the number of carboxylate functions per saccharide unit, said carboxylate functions optionally being carboxylate functions that are naturally present on the saccharide units, being resulting from substitution with radicals -[A]-COOH and/or radicals -[AA] and 6≧p≧0.1,
and the acid functions being in the form of salts of alkali metal cations chosen from the group consisting of Na₊ and K₊.
